(19) United States
(12) Patent Application Publication
(10) Pub. No.: US 2025/0049879 A1
(43) Pub. Date: Feb. 13, 2025

(54) COMPOSITIONS COMPRISING DIMOCARPUS EXTRACT FOR USE IN THE TREATMENT OR PREVENTION OF AN INFECTION CAUSED BY AN ENVELOPED VIRUS

(71) Applicant: PM GROUP COMPANY LIMITED, Bangkok (TH)

(72) Inventors: Prayudh MAHAGITSIRI, Bangkok (TH); Chalermchai MAHAGITSIRI, Bangkok (TH); Ausana MAHAGITSIRI, Bangkok (TH); Thomas JAKSCHITZ, Innsbruck (AT); Gökhan SENLI, Innsbruck (AT); Günther BONN, Zirl (AT)

(21) Appl. No.: 18/716,764

(22) PCT Filed: Dec. 7, 2022
(86) PCT No.: PCT/IB2022/061868

§ 371 Date: Jun. 5, 2024

(30) Foreign Application Priority Data
Dec. 7, 2021 (EP) 21212834.2

(51) Int. Cl. A61K 36/77; A61K 31/192; A61K 31/197; A61K 31/357; A61K 31/366; A61K 31/375; A61K 31/7024; A61P 31/14; A61P 31/16

(52) U.S. Cl. CPC A61K 36/77; A61K 31/192; A61K 31/197; A61K 31/357; A61K 31/366; A61K 31/375; A61K 31/7024; A61P 31/14; A61P 31/16; A61K 2236/31; A61K 2236/37; A61K 2236/51; A61K 2236/53

(57) ABSTRACT

The present invention relates to a composition comprising or corresponding to an extract obtained from Dimocarpus longan for use in the treatment and/or prevention of a disease caused by an enveloped virus, in particular caused by coronaviruses such as SARS-CoV, MERS-CoV and SARS-CoV-2 or influenza viruses such as influenza A and influenza B and/or at least one symptom thereof.

The present invention relates to a composition comprising or corresponding to an extract obtained from fruits the plant family of Sapindaceae, in particular of the genus Dimocarpus or Litchi for use in the treatment and/or prevention of a disease caused by an enveloped virus, such as an influenza virus, a respiratory synctial virus (RSV), a human parainfluenza virus (HPIV), a human metapneumovirus (HPMV), a rhinovirus or a coronavirus (CoV), in particular caused by coronaviruses such as SARS-CoV, MERS-CoV and SARS-CoV-2 or caused by an influenza virus such as Influenza type A viruses or Influenzae virus type B and/or at least one symptom thereof.

In particular the present invention relates to a composition comprising or corresponding to a Dimocapus longan Lour. extract for use in the treatment or prevention of a severe acute respiratory syndrome coronavirus-2 (SARS-CoV-2) infection and/or at least one symptom of coronavirus disease-19 (COVID-19).

Furthermore, the present invention in particular relates to a composition comprising or corresponding to a Dimocapus longan Lour. extract for use in the treatment or prevention of an influenza virus type A infection (subtypes A(H1N1), A(H1N1)pdm09 and A(H3N2) or a influenza type B infection and/or at least one symptom thereof.

### BACKGROUND OF THE INVENTION

Dimocarpus is a genus belonging to the family Sapindaceae, also known as the soapberry family of flowering plants (Angiospermae) to which the lychee, rambutan, guarana, korlan, pitomba, guinep and ackee also belong. The major characteristics of this genus are trees or shrubs which can grow up to 25-40 meters tall with pinnate leaves. The flowers are seen as large panicles. The edible fruit is 3-5 centimeters long containing a single seed surrounded by a layer of fruit pulp. Dimocarpus is primarily distributed in tropical South and Southeast Asia, ranging from Sri Lanka and India to East Malaysia and Australia. The well recognized edible fruits derived from this genus known as “Longan” are produced from Dimocarpus longan Lour., in particular Dimocarpus longan ssp longan var longan, the commercial Longan.

The term “Dimocarpus” in accordance with the present invention refers to the various species of this genus such as Dimocarpus australicus, Dimocarpus dentatus, Dimocarpus foveolatus, Dimocarpus gardneri, Dimocarpus fumatus Dimocarpus confinis, Dimocarpus leichhardtii and Dimocarpus yunnanesis, however, in particular to the species Dimocarpus longan Lour., and its subspecies Dimocarpus longan ssp longan and Dimocarpus longan ssp malesianus and variants.

A further genus of the sapindaceae that may be used in accordance with the invention is Litchi with its sole member Litchi chinensis (Lychee), or Nephelium such as Nephelium lappaceum (Rambutan).

Longan is historically planted as an edible fruit but can also be used for medicinal purposes. Longan fruit contains significant amounts of bioactive compounds such as proteins, carbohydrates, vitamin C, polysaccharides, polyphenols such as corilagin, ellagic acid and its conjugates, 4-O-methylgallic acid, flavone glycosides, glycosides of quercetin and kaempferol, ethyl gallate 1-β-O-galloyl-d-glucopyranose, grevifolin and 4-O-α-I-rhamnopyranosyl-ellagic acid as well as GABA and tannic acid. The fruit has been used in the traditional Chinese medicinal formulation, serving as an agent in relief of neural pain and swelling (Yang et al., Food Research International 44(7):1837-1842 (2011); Zhang et al., Food Science and Human Wellness 9: 95-102 (2020)). Longan fruit can be consumed in many forms of products such as dried Longan pulp, Longan juice, Longan jelly, Longan wine and canned Longan in syrup. Current reports show that Polyphenols and polysaccharides in Longan pulp and pericarp contribute to antioxidant, antiglycation, antityrosinase, potent immunomodulatory and anticancer activities (N. Nuengchamnong & K. Ingkaninan, Food Chem 118: 147-152 (2010); Khan et al., J. Food Sci. Technol. 55: 4782-4791 (2018)). A review of the bioactive compounds and biocativities of longan pulp is provided i.a. in Zhang et al., Food Science and Human Wellness 9: 95-102 (2020). The Longan fruit contains about 83% water, 15% carbohydrates and 1% proteins.

The family of enveloped viruses includes many of the most dangerous pathogenic viruses for humans and livestock, such as, e.g., human immunodeficiency virus (HIV), hepatitis B virus (HBV), hepatitis C virus (HBC), and influenza virus (see, e.g., Rey FA et al., Cell 172(6): 1319-34 (2018) or Vaney MC et al., Cell Microbiol 13(10): 1451-9 (2011)) as well as the Severe Acute Respiratory Syndrome coronavirus 1 (SARS-CoV-1), the Middle East Respiratory Syndrome coronavirus (MERS-CoV) and the Severe Acute Respiratory Syndrome coronavirus 2 (SARS-CoV-2). SARS-CoV-2 is a positive-sense single stranded RNA virus, part of the beta coronaviruses family (Cheng & Shan, Infection 48: 155-163 (2020)). Phylogenetically is 97% related to bats coronavirus, 79% to the Severe Acute Respiratory Syndrome coronavirus-1 (SARS-CoV-1), and 50% to the Middle East Respiratory Syndrome coronavirus (MERS-CoV) (Lu et al., Lancet 395 10224: 565-574 (2020); Zhou et al., Respiratory Research 21: 224 (2020); Perlman, N Engl J Med 382: 760-762 (2020)). Coronaviruses are enveloped, positive-sense, single stranded RNA viruses that are distributed broadly among humans which cause respiratory, enteric, hepatic, and neurologic diseases, in particular frequently mild respiratory infections in humans.

Betacoronaviruses (β-CoVs or Beta-CoVs) are one of four genera of coronaviruses of the subfamily Orthocoronavirinae in the family Coronaviridae, of the order Nidovirales. They are enveloped, positive-sense, single-stranded RNA viruses of mostly zoonotic origin. The genome of betacoronaviruses encodes four structural proteins, known as the S (spike), E (envelope), M (membrane), and N (nucleocapsid) proteins. The N protein holds the RNA genome, and the S, E, and M proteins together create the viral envelope. The spike protein is the major glycoprotein on the coronavirus surface and is responsible for allowing the virus to attach to and fuse with the membrane of a host cell. The spike protein forms a crown-like structure on the surface of a coronaviruses.

The emergence in late 2019 of a novel SARS-CoV causing COVID-19 (Coronavirus disease 19) has given rise to an unprecedented global public health emergency with significant societal and economic ramifications.

On Jan. 31, 2020, after the exponential increase in cases around the world, the World Health Organization (WHO) declared the Severe Acute Respiratory Syndrome virus-2 (officially named as SARS-CoV-2) and its disease COVID-19 as a pandemic (Mahase, BMJ 12: 368(2020)).

Due to the contagiousness of SARS-CoV-2 and the rapid spread, the WHO has declared the ongoing pandemic COVID-19 as a global emergency in March of 2020. As of Jun. 1, 2021, more than 171 million SARS-CoV-2 cases have been confirmed with more than 3,55 million deaths world-wide.

SARS-CoV-2 has a very high degree of similarity to SARS-CoV and MERS-CoV, and indeed analogous receptors are used by these viruses in order to enter the cell and they can therefore replicate in similar tissues (Wu et al., Cell Host Microbe 27: 325-328, (2020)). So far, it has been shown that the structural spike (S) glycoprotein has a very high affinity to the angiotensin converting enzyme 2 (ACE2) receptor, which is ubiquitously expressed in nasal epithelium, lung, heart, kidney and intestine, but is rarely found on immune cells (Wrapp et al., Science 367: 1260-1263, (2020) and Ziegler et al., Cell 181: 1016 (2020)).

Early events occurring directly after SARS-CoV-2 transmission to respiratory tissues can influence the outcome in the context of disease severity—in some patients, infection with COVID-19 results in excessive activation of the immune response at epithelial/immune barriers and the generation of a pro-inflammatory milieu (Magro, Virus Res 286: 198070 (2020) and Zhu et al., N Engl J Med 328: 727-733 (2020)). The development of a cytokine storm and acute lung injury, causing acute respiratory distress syndrome (ARDS), are potential undesirable consequences of the disease. ARDS accompanied by systemic coagulopathy are critical aspects of morbidity and mortality in COVID-19 (Tang et Ia., J Thromb Haemostst 18: 844-847 (2020) and Wang et al., JAMA 323:1061-1069 (2020)). These overshooting immune responses triggered by incoming viruses result in extensive tissue destruction during severe cases, resulting in tissue injury and multi-organ failure (Cheng et al., J Clin Invest; 130(5):2620-2629 (2020) and Huang et al, Lancet 395: 497-506 (2020)). Complement may be among the factors responsible for the immune overactivation, since complement deposition and high anaphylatoxin serum levels have been reported in patients with severe/critical disease (Jodele & Köhl, Br J Pharmacol; 178:2832-2848 (2020)).

As recently shown in transcriptome analyses of bronchoalveolar lavages of patients, the complement system was among the most significantly upregulated intracellular pathways following SARS-CoV-2 infection (Yang et al, Res Sq preprint January (2020) and Sci Immunol. 2021 Apr. 7; 6(58)). In addition, the transcriptomes of primary normal human bronchial epithelial (NHBE) cells infected in vitro with SARS-CoV-2 revealed an enriched complement signature (Yang, et al, supra). Very recently, Ramlall et al. identified in addition to type I IFN and IL-6-dependent inflammatory responses, a robust engagement of complement and coagulation pathways following SARS-CoV-2 infection (Ramlall et al., Nat Med 26: 1609-1615 (2020)).

Influenza viruses of the family Orthomyxoviridae are enveloped negative-strand RNA viruses with segmented genomes. Of two genera, one includes influenza A and B viruses, and the other influenza C virus. The three virus types differ in host range and pathogenicity. A and B type viruses contain eight discrete gene segments, each coding for at least one protein. They are covered with projections of three proteins: hemagglutinin (HA), neuraminidase (NA), and matrix 2 (M2). Each influenza RNA segment is encapsidated by nucleoproteins to form ribonucleotide-nucleoprotein complexes. Types B and C influenza viruses are isolated almost exclusively from humans. Influenza A viruses, however, all circulate within or are derived from an avian reservoir, but can infect a wide variety of warm-blooded animals as well, including not only humans but also swine, horses, dogs, cats, and other mammals. Aquatic birds serve as the natural reservoir for all known subtypes of influenza A virus and probably are the ultimate source of human pandemic influenza strains. Influenza A viruses are subdivided by serologic or genetic characterization of the HA and NA surface glycoproteins that project from the virion. Sixteen HA (or “H”) and 9 NA (or “N”) subtypes are known, abbreviated H1-H16 and N1-N9. (Taubenbeger and Morens, Public Health Rep. 2010; 125(Suppl 3): 16-26). The seasonal influenza A and B viruses are particularly relevant for humans. Influenza A subtypes A(H1N1)pdm09, A(H3N2) and influenza B viruses have been circulating in the human population since 2009. The influenza A(H1N1) virus circulating before the 2009 influenza pandemic has since been completely displaced by the A(H1N1)pdm09 virus. Influenza A(H3N2) viruses can infect birds and mammals. Symptoms of influenza infections comprise e.g. body aches and pains, fever, chills, fatigue, diarrhea and vomiting.

Adults ages 65 and over, children under 5, pregnant people, individuals with underlying chronic medical conditions, such as asthma, diabetes, or heart disease and people with a weakened immune system due to medication (steroids, chemotherapy) or a medical condition (HIV, leukemia) have a higher risk of severe courses of influenza.

A research group at the institute of Hygiene and Medical Microbiology, Medical University of Innsbruck, Austria recently illustrated in standardized human 3D respiratory models (Zaderer et al., Cells 8:1292 (2019); Chandorkar et al., Sci Rep 7:11644 (2017)) that in primary NHBE cells, SARS-CoV-2 infection resulted in extensive tissue destruction and this was associated with intracellular complement activation in epithelial cells and massive anaphylatoxin production (Posch et al, Journal of Allergy and Clinical Immunology,; 147:2083-2097 (2021)).

### SUMMARY OF THE INVENTION

Symptoms of SARS-CoV-2 infection are nonspecific. The most common ones on onset of the infection are fever, weakness and dry cough. Less common symptoms include headache, myalgia, fatigue, oppression in the chest, dyspnea, sputum production, diarrhea, confusion, sore throat, rhinorrhea, chest pain, nausea and vomiting. Up to 50% of patients develop shortness of breath. Severe COVID-19 is characterized by acute respiratory distress syndrome (ARDS) and extensive damage to the alveoli in the lung parenchyma.

The percentage of patients requiring ARDS treatment is about 10% for those who are hospitalized and symptomatic. Some patients are known to be asymptomatic carriers of the infection showing no clinical signs. Usually, severe patients are older and have chronic diseases, among those the most common associated diseases in severe cases are hypertension and cardiovascular diseases.

There thus remains a need for well tolerated therapeutic agents that are effective in the prevention and/or treatment of COVID-19 and secondary symptoms caused by this disease.

This need is addressed by the present invention.

Accordingly, the present invention relates to a composition comprising or corresponding to a Litchi or Dimocarpus extract for use in the treatment and/or prevention of a SARS-CoV-2 infection and/or at least one symptom of COVID-19.

The present invention specifically relates to a composition comprising or corresponding to a Dimocarpus extract for use in the treatment and/or prevention of a SARS-CoV-2 infection and/or at least one symptom of COVID-19.

The present invention also relates to a method for the treatment and/or prevention of a SARS-CoV-2 infection and/or at least one symptom of COVID-19 comprising administering to a subject a therapeutically or prophylactically effective amount of a composition comprising or corresponding to a Litschi or Dimocarpus extract. In this respect, the subject is in general a human.

The present invention specifically relates to a method for the treatment and/or prevention of a SARS-CoV-2 infection and/or at least one symptom of COVID-19 comprising administering to a subject a therapeutically or prophylactically effective amount of a composition comprising or corresponding to a Dimocarpus extract. In this respect, the subject is in general a human.

In particular the extract is a Dimocarpus longan Lour. extract.

In a further embodiment the present invention specifically relates to a method for the treatment and/or prevention of an influenza virus infection and/or at least one symptom of an influenza virus infection comprising administering to a subject a therapeutically or prophylactically effective amount of a composition comprising or corresponding to a Dimocarpus extract. In this respect, the subject is in general a human.

In particular the influenza virus is selected from influenza virus Type A and influenza virus type B. The influenza virus Type A is in particular influenza virus subtype H3N2.

In particular the extract is a Dimocarpus longan Lour. extract.

### BRIEF DESCRIPTION OF THE FIGURES

The invention is also described by the following illustrative figures. The appended figures show:

FIG. 1:

Schematic illustration of the standardized human 3D respiratory model used in the in vitro tests of the examples

The human in vitro model reconstitutes to a large extent the entire upper air way epithelium.

Cells are grown on permeable filter supports at an air-liquid interphase, which allows the cells to get nutrients from the bottom of the dish, where the medium is (like the access in vivo to the blood stream). On the top the cells are exposed to air which induces the differentiation of the cells and the production of mucus to protect them from drying out.

The culture contains mucus producing goblet cells, ciliated epithelial cells and basal cells which have stem cell character. The culture system therefore allows experimental access and manipulation from both sides. They can, for instance, be infected or treated from top (apical side) or medium can be collected for marker analyses from the bottom (basal side).

FIGS. 2a-c:

### SARS-CoV-2

Study of the transepithelial electrical resistance (TEER) in Dimocarpus extract exposed 3D cultures of NHBE cells. TEER measurements are performed by applying an AC electrical signal across electrodes placed on both sides of a cellular monolayer on a permeable filter support and measuring voltage and current to calculate the electrical resistance of the barrier.

FIG. 2a:

Influence of apical and basolateral application of 0.1% and 1% extract on TEER

FIG. 2b:

Measurement of TEER Day 1 post infection (d1PI); 0.1%, 1% and 2% extract

(w/w) apical

FIG. 2c:

Measurement of TEER Day 2 post infection (d2PI); 0.1%, 1% and 2% extract

(w/w) apical

See example 2

FIG. 3:

SARS-CoV-2

Complement downregulation of innate immune response C3a

See example 3

FIG. 4:

SARS-CoV-2

Reduction of infection by Dimocarpus extract in primary NHBE mono layers

See example 4

FIG. 5:

SARS-CoV-2

Reduction of infection by Dimocarpus extract in 3D cultures of NHBE cells

See example 4

FIG. 6:

FIG. 6 provides a schematic illustration of the process for preparation of the Dimocarpus extract to be used in the present invention

FIGS. 7a to 8d

### Influenza Virus

Study of the transepithelial electrical resistance (TEER) in Dimocarpus extract exposed 3D cultures of NHBE cells. TEER measurements are performed by applying an AC electrical signal across electrodes placed on both sides of a cellular monolayer on a permeable filter support and measuring voltage and current to calculate the electrical resistance of the barrier.

See example 5

FIG. 7a:

Measurement of TEER Day 1 post infection (d1PI); influenza A(H3N2) added at MOI 0.05; 1% extract (w/w) apical

FIG. 7b:

Measurement of TEER Day 2 post infection (d2PI); influenza A(H3N2) added at MOI 0.05; 1% extract (w/w) apical

FIG. 7c:

Measurement ofTEER Day 1 post infection (d1PI); influenza B added at MOI 0.05; 1% extract (w/w) apical

FIG. 7d:

Measurement ofTEER Day 2 post infection (d2PI); influenza B added at MOI 0.05; 1% extract (w/w) apical

FIG. 8a:

Measurement of TEER Day 1 post infection (d1PI); influenza A(H3N2) added at MOI 0.005; 1% extract (w/w) apical

FIG. 8b:

Measurement of TEER Day 1 post infection (d1PI); influenza A(H3N2) added at MOI 0.005; 1% extract (w/w) apical

FIG. 8c:

Measurement ofTEER Day 1 post infection (d1PI); influenza B added at MOI 0.005; 1% extract (w/w) apical

FIG. 8d:

Measurement ofTEER Day 2 post infection (d2PI); influenza B added at MOI 0.005; 1% extract (w/w) apical

FIGS. 9a to 10c:

Study of apically and basolaterally released influenza virus particles, analysis by Reverse Transcription Polymerase Chain Reaction (RT-PCR)

See example 6

FIG. 9a:

RT-PCR apical, influenza A(H3N2) added at MOI 0.005; Day 1 post infection (d1PI)

FIG. 9b:

RT-PCR apical, influenza A(H3N2) added at MOI 0.005; Day 2 post infection (d2PI)

FIG. 9c:

RT-PCR apical, influenza B added at MOI 0.005; Day 1 post infection (d1PI)

FIG. 9d:

RT-PCR apical, influenza B added at MOI 0.005, Day 2 post infection (d2PI)

FIG. 10a:

RT-PCR basolateral, influenza A(H3N2) added at MOI 0.005, Day 1 post infection (d1PI)

FIG. 10b:

RT-PCR basolateral, influenza A(H3N2) added at MOI 0.005, Day 2 post infection (d2PI)

FIG. 10c:

RT-PCR basolateral, influenza B added at MOI 0.005; Day 2 post infection (d2PI)

### DETAILED DESCRIPTION OF THE INVENTION

In the context of the present invention, it was surprisingly found that components derived from plants of the family Sapindaceae, such as Litchi and Dimocarpus, in particular of the genus Dimocarpus and even more particular from the species Dimocarpus longan Lour. prevents/inhibits SARS-CoV-2 virus tissue damage, inflammation and infection, as well as influenza virus tissue damage, inflammation and infection.

In particular, it has been surprisingly found that the local application of a composition comprising or corresponding to a Dimocarpus extract enhances mucociliary clearance (MCC) in SARS-CoV-2 infected NHBE cells. MCC is the primary innate defense mechanism of the lung.

The functional components are the protective mucous layer, the airway surface liquid layer, and the cilia on the surface of ciliated cells. The cilia are specialized organelles that beat in metachronal waves to propel pathogens and inhaled particles trapped in the mucous layer out of the airways.

In addition, it has been surprisingly found that the local application of a composition comprising or corresponding to a Dimocarpus extract stabilizes transepithelial electrical resistance (TEER) in SARS-CoV-2 infected NHBE cells. TEER measurement is used to assess the integrity and stability (i.e. the barrier function) of epithelial cells layers.

Furthermore, it has been surprisingly found that the local application of a composition comprising or corresponding to a Dimocarpus extract inhibits complement activation and down regulates the inflammatory markers and chemo attractants for immune cells in SARS-CoV-2 infected NHBE cells.

Additionally, it has been surprisingly found that the infection with SARS-CoV-2 has been inhibited as such.

Moreover, it has been surprisingly found that the local application of a composition comprising or corresponding to a Dimocarpus extract stabilizes transepithelial electrical resistance (TEER) in influenza virus type A(H3N2) and type B virus infected NHBE cells. TEER measurement is used to assess the integrity and stability (i.e. the barrier function) of epithelial cells layers.

Additionally, it has been surprisingly found that the intracellular formation of new influenza type A(H3N2) and type B viral particles and their excretion has been prevented.

Glycoproteins on lipid bi-layered virus surfaces act as an access key and allow the virus to enter the cell, it a has been surprisingly found that components of the Dimocarpus extract present in the composition for use according to the present invention cover the surface proteins of the virus and thus hinder that virus enters the cells.

The present invention thus in particular provides the following:
(1) A composition comprising or corresponding to a Dimocarpus extract for use in the treatment and/or prevention of a respiratory infection with an enveloped virus, enveloped single stranded virus, enveloped positive single strand RNA virus (+ssRNA) virus, an influenza or a coronavirus and/or at least one symptom thereof.
(2) The composition for use according to (1) in the treatment of a respiratory infection with an enveloped virus, enveloped single stranded virus, enveloped positive single strand RNA virus (+ssRNA) virus, an influenza or a coronavirus and/or at least one symptom thereof.
(3) The composition for use according to (1) in the prevention of a respiratory infection with an enveloped virus, enveloped single stranded virus, enveloped positive single strand RNA virus (+ssRNA) virus, an influenza or a coronavirus and/or at least one symptom thereof.
(4) The composition for use according to any one of (1) to (3), wherein the respiratory infection is an upper and/or lower respiratory tract infection.
(5) The composition for use according to any one of (1) to (4), wherein the Dimocarpus extract is an extract of Dimocarpus longan Lour.
(6) The composition for use according to any one of (1) to (5) above, wherein the virus is selected from an influenza virus, a respiratory syncytial virus (RSV), a human parainfluenza virus (HPIV) a human metapneumovirus (HPMV), a rhinovirus or a coronavirus (CoV).
(7) The composition for use according to any one of (1) to (6), wherein the virus is selected from SARS-CoV, MERS-CoV and SARS-CoV-2.
(8) The composition for use according to any one of (1) to (7), wherein the infection is a SARS-CoV-2 infection (COVID-19).
(9) The composition for use according to any one of (1) to (6), wherein the virus is selected from a subtype of influenza virus A (in particular A(H3N2) or influenza virus type B
(10) The composition for use according to any one of (1) to (6) and (9), wherein the infection is an influenza virus A(H3N2) or influenza virus B infection.
(11) The composition for use according to any one of (1) to (10), wherein the Dimocarpus extract comprises one or more of vitamin C, one or more polyphenols selected from corilagin, gallic acid, ellagic acid, ellagic acid conjugates, (−)-epicatechin, quercetin, kaempferol, tannic acid (tannin) and chlorogenic acid; protocatechuic acid, brevifolin, γ-aminobutyric acid (GABA), carbohydrates; and water.
(12) The composition for use according to any one of (1) to (11), wherein the Dimocarpus extract comprises vitamin C, one or more polyphenols selected from corilagin, gallic acid, ellagic acid, ellagic acid conjugates, (−)-epicatechin, quercetin, kaempferol, tannic acid (tannin) and chlorogenic acid; protocatechuic acid, brevifolin, γ-aminobutyric acid (GABA), carbohydrates and water.
(13) The composition for use according to any one of (1) to (12), wherein the Dimocarpus extract comprises vitamin C, corilagin, gallic acid, ellagic acid, ellagic acid conjugates tannic acid, GABA, saccharose, glucose, fructose, polysaccharides and water.

In the context of the present invention the Dimocarpus extract comprises 100-1000 mg/kg, preferably 105-760 mg/kg, or 400-1000 mg/kg, more preferably 600-760 mg/kg and most preferably 720 mg/kg vitamin C.

In the context of the present invention the Dimocarpus extract comprises 750-2000 mg/kg, 750-1800 mg/kg, preferably 1188-1880 mg/kg, more preferably 1200-1500 mg/kg and most preferably 1250 mg/kg corilagin.

In the context of the present invention the Dimocarpus extract comprises 200-600 mg/kg, preferably 340-428 mg/kg, more preferably 380-430-mg/kg and most preferably 409 mg/kg gallic acid.

In the context of the present invention the Dimocarpus extract comprises 600-1250 mg/kg, 600-1200, preferably 1010-1230 mg/kg, more preferably 1010-1100 mg/kg and most preferably 1050 mg/kg ellagic acid (including ellagic acid conjugates).

In the context of the present invention the Dimocarpus extract comprises 200-700 mg/kg, preferably 420-510 mg/kg, more preferably 450-480 mg/kg and most preferably 430 mg/kg tannic acid.

In the context of the present invention the Dimocarpus extract comprises 1200-2000 mg/kg, preferably 1133-1896 mg/kg, more preferably 1150-1700 mg/kg and most preferably 1638.30 mg/kg GABA.

In the context of the present invention the Dimocarpus extract comprises 30-50%, preferably 30-45%, more preferably 30-40%, and most preferably 42.4% (w/w) of the total extract saccharose (sucrose).

In the context of the present invention the Dimocarpus extract comprises 5-25%, preferably 7.5-23%, more preferably 10-20%, and most preferably 11.7% (w/w) of the total extract glucose.

In the context of the present invention the Dimocarpus extract comprises 10-20%, preferably 10-18%, more preferably 10-15% and most preferably 13.3% (w/w) of the total extract fructose.

In the context of the present invention the Dimocarpus extract comprises 50-85 g/kg, preferably 50-80 g/kg, more preferably 50-70 g/kg and most preferably 66 g/kg polysaccharides.

In the context of the present invention the Dimocarpus extract comprises 15-25%, preferably 18-24%, more preferably 19-23% and most preferably 21% (w/w) water.

In the context of the present invention the total phenolic content of the Dimocarpus extract is 2950-7600 mg/kg, preferably 3500-7600 mg/kg, more preferably 4000-6500 mg/kg and most preferably 7565 mg/kg.

In the context of the present invention the total carbohydrate content is 700-800 g/kg and preferably 742 g/kg.

The Master thesis “Mono-, Oligo- and Polysaccharide analysis of a beverage obtained from the fruit of Dimocarpus longan” by Tobias Schlappack, Sep. 16, 2020, Institute of Analytical Chemistry and Radiochemistry, Leopold-Franzens University Innsbruck discloses a saccharide profile regarding the free saccharides, oligo- and polysaccharides as well as the total carbohydrate amount and the water content of a beverage obtained from the fruit of Dimocarpus longan.

According to the present invention the composition for use has a sugar content of about 74 to 84° Brix, more preferred a sugar content of about 76 to 82° Brix and most preferred a sugar content of about 78 to 80° Brix.

In one embodiment the present invention provides the composition for use according to any one of (1) to (13), wherein the Dimocarpus extract, in particular the Dimocarpus longan Lour. extract comprises:

| | | |
|---|---|---|
| Vitamin C | 400-1000 | mg/kg |
| Tannic acid | 200-700 | mg/kg |
| Gallic acid | 200-600 | mg/kg |
| Ellagic acid (incl. conj.) | 600-1200 | mg/kg |
| Corilagin | 750-1800 | mg/kg |
| GABA | 1200-2000 | mg/kg |
| Sucrose | 30-50% | (w/w) |
| Glucose | 5-25% | (w/w) |
| Fructose | 10-20% | (w/w) |
| Water | 15-25% | (w/w) |

Thus, the present invention particularly provides:
(14) The composition for use according to any one of (1) to (13), wherein the Dimocarpus extract, in particular the Dimocarpus longan Lou.r extract comprises

| | | |
|---|---|---|
| Vitamin C | 100-1000 | mg/kg |
| Tannic acid | 200-700 | mg/kg |
| Gallic acid | 200-600 | mg/kg |
| Ellagic acid (incl. conj.) | 600-1200 | mg/kg |
| Corilagin | 750-2000 | mg/kg |
| GABA | 1200-2000 | mg/kg |
| Total phenolic content | 2950-7600 | mg/kg |
| Total carbohydrate | 700-800 | g/kg |
| Sucrose | 30-50% | (w/w) |
| Glucose | 5-25% | (w/w) |
| Fructose | 10-20% | (w/w) |
| Polysaccharides | 50-85 | g/kg |
| Water | 15-25% | (w/w) |

(15) The composition for use according to any one of (1) to (13), wherein the Dimocarpus extract, in particular the Dimocarpus longan Lour. extract comprises

| | | |
|---|---|---|
| Vitamin C | 105-760 | mg/kg |
| Tannic acid | 420-510 | mg/kg |
| Gallic acid | 340-428 | mg/kg |
| Ellagic acid (incl. conj.) | 1010-1230 | mg/kg |
| Corilagin | 1188-1880 | mg/kg |
| GABA | 1133-1896 | mg/kg |
| Total phenolic content | 3500-6500 | mg/kg |
| Total carbohydrate | 700-800 | g/kg |
| Sucrose | 30-50% | w/w |
| Glucose | 7-23% | w/w |
| Fructose | 10-20% | w/w |
| Polysaccharides | 50-80 | g/kg |
| Water | 15-24% | w/w |

(16) The composition for use according to any one of (1) to (14), wherein the Dimocarpus extract, in particular the Dimocarpus longan Lour. extract, comprises

| | | |
|---|---|---|
| Vitamin C | 200-760 | mg/kg |
| Tannic acid | 420-510 | mg/kg |
| Gallic acid | 380-430 | mg/kg |
| Ellagic acid (incl. conj.) | 1010-1100 | mg/kg |
| Corilagin | 1200-1500 | mg/kg |
| GABA | 1105-1700 | mg/kg |
| Total phenolic content | 4000-7600 | mg/kg |
| Total carbohydrate | 700-800 | g/kg |
| Sucrose | 300-400 | g/kg |
| Glucose | 100-200 | g/kg |
| Fructose | 100-150 | g/kg |
| Polysaccharides | 50-70 | g/kg |
| Water | 19-23% | w/w |

(17) The composition for use according to any one of (1) to (16), wherein the Dimocarpus extract, in particular the Dimocarpus longan Lour. extract, comprises

| Vitamin C | 720 | mg/kg |
|---|---|---|
| Tannic acid | 430 | mg/kg |
| Gallic acid | 409 | mg/kg |
| Ellagic acid (incl. conj.) | 1050 | mg/kg |
| Corilagin | 1250 | mg/kg |
| GABA | 1638 | mg/kg |
| Total phenolic content | (0.76%) 7565 mg/kg | |
| Total carbohydrate | 742 | g/kg |
| Sucrose | 424 | g/kg |
| Glucose | 117 | g/kg |
| Fructose | 133 | g/kg |
| Polysaccharides | 66 | g/kg |
| Water | 21% | w/w |

(18) The composition for use according to any one of (1) to (17), wherein the extract is produced from the whole dried or fresh fruit, the pericarp, the seeds, aril or the pulp of dried or fresh fruit(s) or a combination of at least two of pericarp, seeds, aril, pulp of fresh fruit. The use of whole fresh fruit as the starting material is particularly preferred.
(19) The composition for use according to any one of (1) to (18), wherein the Dimocarpus extract, in particular the Dimocarpus longan Lour. extract, is obtainable by a method comprising the following steps:
   (a) extraction of the Dimocarpus juice from whole fresh fruit, followed by
   (b) separation of the solids from the obtained raw liquid;
   (c) concentration of the liquid obtained in step (b) to obtain a sugar concentration of about 74 to 840 Brix, preferably 76 to 820 Brix and most preferably to 78 to 80° Brix and
   (d) aseptical packing.
(20) The composition for use according to any one of (1) to (19), wherein the Dimocarpus extract is obtainable by a method comprising the following steps:
   (a) Milling whole fresh Dimocarpus fruit;
   (b) Extraction of the juice from the milled whole fruit obtained in (a);
   (c) Conditioning of the raw juice obtained in (b) by rapidly heating to about 95-98° C., maintaining at about 95-98° C. followed by rapid cooling to about 5-15° C.;
   (d) Separation of the supernatant from the product of step (c) by centrifugation and microfiltration;
   (e) Concentration of the supernatant obtained in step (d), preferably by evaporation at reduced pressure to obtain a sugar concentration of about 74-84° Brix;
   (e) Microfiltration of the concentrate obtained in step (e); and
   (f) aseptical packing.
(21) The composition for use according to any one of (1) to (20) wherein the composition is to be applied topically via the ocular, nasal, or the (naso)pharyngeal route, i.e. for the topical application to conjunctival epithelia of the eye and the epithelia of the upper respiratory tract.
(22) The composition for use according to any one of (1) to (21), in form of a mouthwash, gargle, nasal drops, nasal spray/aerosol, pharyngeal drops, or a pharyngeal spray/aerosol.
(23) The composition according to any one of (1) to (21), in form of nasal spray/aerosol or a pharyngeal spray/aerosol.

In the context of the present invention “Dimocarpus” (also referred to herein as “Dimocarpus spec.”) may, for example, be selected from the group of Dimocarpus longan, Dimocarpus australianus, Dimocarpus dentatus, Dimocarpus foveolatus, Dimocarpus fumatus, Dimocarpus gardneri, Dimocarpus confinis, Dimocarpus leichhardtii and Dimocarpus yunnanesis.

The extract may be an extract obtained from one of the Dimocarpus species/subspecies or from a mixture of at least two of the above.

In the preferred embodiments of the composition of the invention, the Dimocarpus extract is an extract of Dimocarpus longan Lour.

In the more preferred embodiment of the invention the extract is prepared from Dimocarpus longan Lour. to be used for the prevention of COVID-19 in form of a nasal spray, nasopharyngeal or pulmonary spray. The use in form of a nasal spray is most preferred.

In a further, more preferred embodiment of the invention the extract is prepared from Dimocarpus longan Lour.to be used for the prevention of an influenza virus infection, in particular of an influenza virus type A(H3N2) or influenza virus type B infection in form of a nasal spray, nasopharyngeal or pulmonary spray. The use in form of a nasal spray is most preferred.

### Composition According to the Invention and its Preparation

The term “extract” means any substance or derivative product or mixture of components that can be obtained from Dimocarpus (the Dimocarpus extract) by any appropriate method known to the person of skills in the art.

The extract can be obtained from all the constituents of the whole plant such as leaves, bark, flowers, seeds, pericaps, fruits, pulp, aril, stalks, branches, stems, roots and wood, as well as parts thereof. Fresh or dried fruit may be used. Different Dimocarpus constituents/parts can be used individually or together.

The use of whole fresh fruit is particularly preferred.

It is understood that any solid parts/particles such as the fruit shell, seed coat, pericarp and solid components of the fruit pulp are separated from the liquid juice in the course of the process by means of juicing/pressing, sedimentation/centrifugation and (micro)filtration.

In particular, the process for producing the Dimocarpus extract in accordance with the invention comprises the steps as disclosed in (19) and (20), above.

As used herein the term “juice extraction” refers to a process whereby the liquid part is separated from the solid parts of the fruit.

It is preferred that the whole Longan fruits are milled prior to extraction, by means known to a person of skill in the art, e.g. by a hammer mill.

Methods thus include solvent extraction, but also means other than solvent extraction, such as a (cold) pressed juice (fresh juice) obtained from fresh plant material by methods known to a person of skill in the art such using hydraulic presses (e.g., standard hydraulic cold-press technology with vertical pressing layers), roll mills or double screw juicers or a juice extractor, in particular an industrial scale juice extractor. The use of an industrial scale juice extractor is particularly preferred. Concentration methods comprise direct heating, steam heating and vacuum evaporation, whereby vacuum evaporation is particularly preferred.

According to a preferred embodiment of the present invention, the components obtained from Dimocarpus are obtained by a process comprising the following steps:
(1) milling fresh whole Dimocarpus longan (Longan fruit); preferably using a hammer mill and optionally passing through a 32 mm diameter sieve to remove first solids and lumps to obtain a homogenous mass;
(2) extracting raw fruit juice from the minced fruit mass obtained in step (1) by means of a juice extractor, preferably for about 5 minutes under pressure, preferably of about 0.8 to 4.5 bar;
(3) discarding the paste obtained after extraction and rapidly heating the raw juice obtained in step (2) to about 95° C., maintaining the juice at a temperature of 95° C. to 98° C. for at least 45 minutes, more preferably 98° C. for 45 minutes, followed by cooling rapidly to a temperature of about 5.0 to 15.0° C., more preferably to a temperature of about 12° C.;
(4) subjecting the cold liquid product of step (3) to at least one sediment separation process at 4000 rpm to 7200 rpm; preferably at 7200 rpm and discarding the residue;
(5) subjecting the supernatant obtained in step (4) to a sediment filtration (0.2 micron cut off) at a temperature of 45° C., preferably at a temperature of about 15 to 40° C., more preferably at a temperature of about 12° C., to obtain a turbidity of 40 NTU;
(6) subjecting the filtrate obtained in step (5) to an evaporation step under reduced pressure, wherein the filtrate is heated to a temperature of about 83.0 to 87.0° C., preferably to about 85° C. at least once under reduced pressure, followed by stepwise cooling to a temperature of about 35 to 45° C., preferably to 40° C., to obtain a sugar concentration of about 74 to 84° Brix, preferably of about 76 to about 820 Brix and most preferably of about 78 to 80° Brix;
(7) Optionally blending the concentrate obtained in step (6) to obtain about 76 to about 78° Brix at a temperature of about 35.0 to 45.0° C.;
(8) Filtration of the concentrates (1.0 mm cut off, preferably about 0.15 mm); and
(9) aseptical packing and cooling to a temperature 40° C. to obtain the Dimocarpus extract for use according to the present invention

In a particularly preferred embodiment, the Dimocarpus extract is obtained/obtainable by the process disclosed in the Thai petty patent application number TH 2103000091. (A flowchart of the process is provided in FIG. 6 for illustrative purposes.)

The extract can be obtained from all the constituents of the whole plant such as leaves, bark, flowers, seeds, pericaps, fruits, stalks, branches, stems, roots and wood, as well as parts thereof. Fresh or dried fruit may be used. Different Dimocarpus constituents/parts can be used individually or together.

The use of whole fresh fruit as the starting material is particularly preferred.

### Exemplary Process

It is particularly preferred that the composition comprising or corresponding to the Dimocarpus longan extract for use in accordance with the present invention is prepared by the following process as illustrated in FIG. 6 and disclosed in TH 2103000091.

### Characterization of the Dimocarpus Extract

Qualitative characterization of the Dimocarpus extract of the invention by its components was carried out by ultra-high performance liquid chromatography-quadrupole time-of-flight mass spectrometry (UHPLC-UV-HR-QTOF-MS).

The method used was conducted as follows: Thermo Scientific Dionex UltiMate 3000 coupled with a maxis Impact Ultra High Resolution TOF-MS from Bruker Daltonics was used for the analysis. Agilent RRHD Zorbax C18 (2.1×100 mm, 1.8 μm) column was used for chromatographic separations of gallic acid, ellagic acid (and conjugates), corilagin. The mobile phase was consisted of acetonitrile (B) and 0.2% formic acid (FA) in water (A). The flow rate was 0.4 mL/min, injection volume was 2 μL. The column oven was set at 45° C. The sampler was at 20° C. The LC gradient was as follow:

Min/B %: 0/0, 2/0, 17/50, 19/100, 20.5/100, 2I/O, 23/0. The eluate from LC was directly introduced into the mass spectrometer with mass scanning from 50-2000 m/z and spectra rate 4 Hz, using electrospray ionization in positive mode. The mass accuracy before each run was verified by comparison with sodium formate adducts. The mass accuracies were rounded to 1 mDa, the corresponding retention times to 0.05 min. The UV spectra were recorded at 270 nm. Compass Data Analysis 4.2 from Bruker® was used for the interpretation of the mass signals. Each sample was measured in form of technical triplicates.

The above-mentioned instrumental parameters and LC column were used also for tannins quantification (as gallic acid equivalent) with minor adjustments to the LC method as described below: Min/B %: 0/3, 15/50, 18/80, 19/100, 21/100, 21.5/0, 23/0. The autosampler was maintained at 4° C.

For the analysis of vitamin C, Thermo Scientific Accucore™ HILIC silica column (2.1×150 mm, 2.6 μm) was used. The mobile phase was consisted of acetonitrile (B) and 0.01% formic acid (FA) in water (A). The flow rate was 0.4 mL/min, injection volume was 1 μL. The column oven was set at 25° C. The sampler was at 20° C. The LC gradient was as follow: Min/B %: 0/95, 5/95, 5.5/60, 8/80, 9/15, 13/15, 14/95, 17/95. The eluate from LC was directly introduced into the mass spectrometer (ESI negative) with mass scanning from 50-800 m/z and spectra rate 4 Hz. XIC (m/z 173) was used for the quantification of vitamin C.

For the analysis of GABA Eppendorf BioSpectrometer® basic (Eppendorf, Hamburg, Germany) operating at 340 nm was employed.

In a preferred embodiment, the Dimocarpus extract according to the present invention comprises the following components as shown in Table 1, below.

TABLE 1
Preferred qualitative composition of the Dimocarpus longan
extract of the present invention

| Vitamin C | 720 | mg/kg |
|---|---|---|
| Tannic acid | 430 | mg/kg |
| Gallic acid | 409 | mg/kg |
| Ellagic acid (incl. conj.) | 1050 | mg/kg |
| Corilagin | 1250 | mg/kg |
| GABA | 1638 | mg/kg |
| Total phenolic content | 7565 mg/kg (0.76 % w/w) | |
| Total carbohydrate | 742 | g/kg |
| Sucrose | 424 | g/kg |
| Glucose | 117 | g/kg |
| Fructose | 133 | g/kg |
| Polysaccharides | 66 | g/kg |
| Water | 21% | w/w |

### Pharmaceutical Formulations of the Invention

The compositions comprising or corresponding to the Dimocarpus extract according to the present invention can be administered by any means which causes contact between said extract and the site of action in a mammal's body, preferably being that of a human being, and the form of pharmaceutical formulation which contains them.

The composition/formulation is preferably an aqueous composition.

According to the present invention, pharmaceutical formulations for topical application and the topical application of the formulations according to the present invention are preferred. The topical application to the epithelial lining of the upper respiratory tract, and the eyes is particularly preferred. It is thus preferred that the composition is administered to the subject via the ocular, nasal or the pharyngeal route, in form of form of eye drops, a mouthwash, gargle, nasal spray/aerosol, nasal drops, a pharyngeal spray/aerosol or pharyngeal drops. The administration via the nasal route or the pharyngeal route, is particularly preferred. Accordingly, it is particularly preferred that the aqueous composition is provided in the form of a mouthwash, gargle, nasal spray/aerosol, nasal drops, a pharyngeal spray/aerosol or pharyngeal drops.

As the administration to the to the epithelial lining of the upper respiratory tract is most preferred the application in form of a mouthwash, gargle, nasal spray/aerosol, nasal drops, a pharyngeal spray/aerosol, pharyngeal drops is most preferred. Among the afore mentioned dosage forms, a nasal or pharyngeal spray is most preferred.

Thus, the present invention further provides pharmaceutical formulations/compositions suitable for topical application comprising the Dimocarpus extract according to the present invention as laid out above.

The composition can be formulated by pharmaceutical by techniques known to the person skilled in the art, such as, e.g., the techniques described in “Remington: The Science and Practice of Pharmacy”, Pharmaceutical Press, 22nd edition and Bauer et al., Pharmazeutische Technologie, 5. Edt. Govi-Verlag Frankfurt, 1997; Rudolf Voigt, and Pharmazeutische Technologie, 9. Edt., Deutscher Apotheker Verlag Stuttgart, 2000).

In particular, the composition can be formulated as a dosage form for nasal, pharyngeal (e.g., through mouth and/or nose). Dosage forms for nasal administration include, e.g., a nasal spray (e.g., a nasal pump spray) or nasal drops. Dosage forms for pharyngeal administration include, e.g., a pharyngeal spray or pharyngeal drops.

The Dimocarpus extract of the present invention is used in the pharmaceutical formulation of the invention at pharmaceutically effective concentrations to achieve the desired effect.

As a rule, the formulations/compositions according to the present inventions may comprise 0.1 to 10% (w/w), preferably the compositions according to the present invention comprise 0.5-7% (w/w), more preferably 1-5% (w/w) and most preferably 2-4% (w/w) of the Dimocarpus extract according to the present invention with regard to the total weight of the formulation, preferably as a solution in a liquid physiologically/pharmaceutically acceptable vehicle. Preferably the vehicle is aqueous.

The vehicle/diluent may be selected from the group comprising water (such as water for injections (aqua ad injectabilia), double distilled water (aqua bidist) and purified water (aqua purificata), physiological saline, phosphate buffered saline (PBS) or any other physiologically/pharmaceutically acceptable buffer systems such as Sörensen buffer, sodium citrate/citric acid, glycerol, sorbitol, and mixtures thereof as well as oily vehicles such as sesame oil. The use of aqueous vehicles is preferred.

The use of water for injection, double distilled water, purified water, physiological saline and PBS as a vehicle or mixtures thereof is particularly preferred, whereby the use of water for injection or purified water as a vehicle/diluent is the most preferred.

When formulating aqueous products for the application on epithelial lining, it is critical to control properties such as viscosity, pH value, buffer capacity and osmolality.

The pharmaceutical formulations/compositions according to the present invention, in addition to physiologically acceptable vehicles well known to a person of skill in the art, may thus comprise additional ingredients such as osmolarity/tonicity adjusting agents such as NaCl, sorbitol, glucose and dextrose; pH adjusting agents such as citric acid, sodium hydroxide, hydrochloric acid and sulphuric acid; buffer components such sodium citrate or sodium phosphate and excipients for enhancing the viscosity such as hydroxy ethyl cellulose, traganth, sodium hyaluronate and xanthan.

In particular for nasal application/application to the upper respiratory tract the pH should be adjusted to a range between 3.5 and 7.5, more preferred between 4 and 7.5 and most preferred between 4.5 and 6.5.

The pH of dosage forms for ocular application should preferably be adjusted to a range between 6 and 8.

The pH of the aqueous composition can be adjusted (e.g., to any of the afore mentioned pH ranges or values) using, e.g., sodium hydroxide or hydrochloric acid and/or any other suitable pH adjusting agent(s).

The aqueous composition may have an osmolality of, e.g., about 200 mOsm/kg to about 800 mOsm/kg, preferably an osmolality of about 250 mOsm/kg to about 500 mOsm/kg and more preferably an osmolarity of about 280-500 mOsm/kg.

The osmolality of the aqueous composition can be adjusted (e.g., to any of the afore-mentioned osmolality ranges or values) using, e.g., sodium chloride and/or any other suitable osmolality adjusting agent(s).

Unless specifically indicated otherwise, all properties and parameters referred to herein, including any pH values as well as any amounts/concentrations (indicated, e.g., in mg/ml, in % w/v or in % v/v), are preferably to be determined at standard ambient temperature and pressure conditions, particularly at a temperature of 25° C. (298.15 K) and at an absolute pressure of 101.325 kPa (1 atm). Accordingly, it is preferred that any pH indicated herein is to be determined at a temperature of 25° C., more preferably at a temperature of 25° C. and an absolute pressure of 1 atm.

As used herein, unless explicitly indicated otherwise or contradicted by context, the terms “a”, “an” and “the” are used interchangeably with “one or more” and “at least one”. Thus, for example, a composition comprising “an” excipient can be interpreted as referring to a composition comprising “one or more” excipients.

It is to be understood that wherever numerical ranges are provided/disclosed herein, all values and subranges encompassed by the respective numerical range are meant to be encompassed within the scope of the invention. Accordingly, the present invention specifically and individually relates to each value that falls within a numerical range disclosed herein, as well as each subrange encompassed by a numerical range disclosed herein.

In the context of the present invention the term “total phenolic content” relates to all phenolic compounds which have been determined by the Folin-Ciocalteu assay, including but not limited to the phenolic compounds specifically identified.

In the context of the present invention the term “total carbohydrate” relates to all carbohydrate compounds, including the polysaccharides which have been determined by a phenol sulphuric acid spectroscopic assay, including but not limited to the carbohydrate compounds specifically identified.

Further suitable dosage forms for the anti-viral pharmaceutical formulations according to the present invention are orally applicable dosage forms such as hard and soft candy, dragées, pastilles, (throat) lozenges and (medicinal) chewing gums allowing the direct contact of the Dimocarpus longan extract of the present invention with the epithelial lining of the upper respiratory tract/the oral cavity, the portal of entry for the pathogen.

Thus, in a further embodiment the Dimocarpus longan extract of the present invention may be incorporated in soft or hard candy, pastilles, (throat) lozenges or (medicinal) chewing gums using techniques and carriers, excipients and additives well known to a person skilled in the art.

The formulation examples below are included for illustrative purposes only and shall not limit the scope of the invention.

### Formulation Examples

The following compositions/formulations are particularly preferred for use in accordance with the present invention:

(a)
Dimocarpus longan extract according to the present invention, in particular in accordance with Table 1 0.1-10% (w/w)
Glycerine 32% (w/w)
Aqueous Sorbitol solution (70% (w/w)) 39.1-50% (w/w)
Purified water 15% (w/w)
   (b)
Dimocarpus longan extract according to the present invention, in particular in accordance with Table 1 0.1-10% (w/w)
Propolis 0.1% (w/w)
Licorice powder 0.13% (w/w)
Peppermint powder 0.13% (w/w)
Glycerin 1.97% (w/w)
Citric acid 0.99% (w/w)
Disodium ethylenediaminetetraacetate 0.013% (w/w)
Hydroxypropyl cellulose 0.99% (w/w)
Sodium chloride 0.66% (w/w)
Purified water 85.017%-94.917% (w/w) (i.e. ad 100% (w/w))

The term ad 100% (w/w) in the context of the present invention indicates that purified water is added until the envisaged final total weight of the formulation has been reached.

(c)

Dimocarpus longan extract according to the present invention, in particular in accordance with Table 1 0.1-10% (w/w)
in aqua bidist. or physiological saline

The above formulations may in particular be used for administration by the pharyngeal and nasal route, such as in form of nasal drops, nasal spray/aerosol, pharyngeal drops or pharyngeal spray/aerosol.

As stated above, further suitable dosage forms for the anti-viral pharmaceutical formulations according to the present invention are orally applicable dosage forms such as hard and soft candy, dragées, pastilles, (throat) lozenges and (medicinal) chewing gums allowing the direct contact of the Dimocarpus longan extract of the present invention with the epithelial lining of the upper respiratory tract/the oral cavity, the portal of entry for the pathogen.

Thus, in a further embodiment the Dimocarpus longan extract of the present invention may be incorporated in soft or hard candy, pastilles, (throat) lozenges or (medicinal) chewing gums using techniques and carriers, excipients and additives well known to a person skilled in the art. Such dosage forms according to the present invention may comprise the Dimocarpus longan extract of the present invention (i.e. the active ingredient) in an amount of about 0.1 to about 20% (w/w) and the base and further carriers, excipients and additives (the non-active ingredients) in an amount of about 80 to about 99.9% (w/w) of the total weight of the formulation.

### Exemplary Formulations for a Candy, Dragée, Pastille or Lozenge May Comprise the Following:

| | | |
|---|---|---|
| Dimocarpus longan extract | 0.1-20% | (w/w) |
| sugar or sugar substitute | 0-98% | (w/w) |
| filler | 0-98% | (w/w) |
| gum arabic | 0-15% | (w/w) |
| water | 0.1-15% | (w/w) |
| fat | 0-15% | (w/w) |
| natural or artificial flavoring | 0.01-10% | (w/w) |

### Exemplary Formulations for a (Medicinal) Chewing Gum May Comprise the Following:

| | | |
|---|---|---|
| Dimocarpus longan extract | 0.1-20% | (w/w) |
| polyisobutylene | 0-50% | (w/w) |
| polyvinylacetate | 0-50% | (w/w) |
| natural gum such as chicle | 0-50% | (w/w) |
| sugar or sugar substitute | 20-80% | (w/w) |
| filler | 0-98% | (w/w) |
| water | 0-15% | (w/w) |
| fat | 0-15% | (w/w) |
| natural or artificial flavoring | 0.01-10% | (w/w) |

In a preferred embodiment The Dimocarpus longan extract according to the present invention, is a Dimocarpus longan extract in accordance with Table 1, above.

### Indications

In the context of the present invention the pharmaceutical formulation according the present invention may be used in a therapeutic method of treating and/or preventing a respiratory infection with an enveloped virus, preferably with an enveloped single stranded virus, more preferably with an enveloped positive single strand RNA virus (+ssRNA) virus, even more preferably with an influenza or a coronavirus and/or at least one symptom thereof.

In the context of the present invention the enveloped virus is particularly selected from an influenza virus, a respiratory syncytial virus (RSV), a human parainfluenza virus (HPIV) a human metapneumovirus (HPMV), a rhinovirus or a coronavirus (CoV).

With regard to infections caused by a coronavirus, the treatment and/or prevention of infections caused by SARS-CoV, MERS-CoV and SARS-CoV-2 are particularly preferred.

Thus, the present invention in particular provides a composition comprising or corresponding to a Dimocapus extract for use in the treatment and/or prevention of a severe acute respiratory syndrome coronavirus-2 (SARS-CoV-2) infection and/or at least one symptom of coronavirus disease-19 (COVID-19).

In a further particular preferred embodiment, the present invention provided a composition comprising or corresponding to a Dimocapus extract for use in the treatment of a severe acute respiratory syndrome coronavirus-2 (SARS-CoV-2) infection and/or at least one symptom of coronavirus disease-19 (COVID-19).

In another particular preferred embodiment, the present invention provides a composition comprising or corresponding to a Dimocapus extract for use in the prevention of a severe acute respiratory syndrome coronavirus-2 (SARS-CoV-2) infection and/or at least one symptom of coronavirus disease-19 (COVID-19).

The symptoms of coronavirus disease-19 referred to above comprise one or more of fever, chills, (dry) cough, congestion or runny nose, fatigue, muscle or body aches, sore throat, diarrhea, nausea or vomiting, conjunctivitis, headache, loss of taste or smell, discoloration on fingers or toes or skin rash, trouble breathing or shortness of breath, constant pain or pressure on the chest, abdominal pain, loss of speech or ability to move, sudden confusion and bluish lips or face.

More severe symptoms and manifestations of coronavirus disease-19 comprise pneumonia, severe pneumonia, pulmonary fibrosis, (acute) lung injury, acute respiratory syndromes such as severe acute respiratory syndrome (SARS) and acute respiratory distress syndrome (ARDS).

Extra-pulmonary symptoms/manifestations comprise hematologic and/or immune system-related manifestations of COVID-19 include various forms of hematological abnormalities, including lymphopenia (a.k.a. lymphocytopenia), leukocytosis, leukopenia, neutrophilia, abnormal blood clotting, dysregulated blood coagulation, thrombocytopenia, pulmonary embolism, disseminated intravascular coagulation, deep vein thrombosis, and prothrombotic state; cardiovascular manifestations of COVID-19 include myocardial injury, acute cardiac injury, acute coronary syndromes (ACS), cardiomyopathy, acute cor pulmonale, cardia arrhythmias (including new-onset atrial fibrillation, heart attack, heart block, and ventricular arrhythmias), cardiogenic shock, myocardial ischemia, acute cor pulmonale, and/or thrombotic complications; renal manifestations of COVID-19 include acute kidney injury (AKI), proteinuria and hematuria; and may be characterized by electrolyte abnormalities (such as hyperkalemia, hyponatremia, and/or hypernatremia); gastrointestinal (GI) manifestations of COVID-19 include diarrhea, nausea, vomiting, abdominal pain, anorexia, anosmia, and dysgeusia as well as hepatobiliary (hepatic) manifestations endocrinologic manifestations of COVID-19, neurologic and ophthalmologic manifestations of COVID-19 and dermatologic manifestations of COVID-19.

With regard to infections caused by an influenza virus, the treatment and/or prevention of infections caused by influenza virus type A (such as A(H3N2) and influenza virus type B are particularly preferred.

Thus, the present invention in particular provides a composition comprising or corresponding to a Dimocapus extract for use in the treatment and/or prevention of influenza virus infection and/or at least one symptom of influenza virus infections.

In a further particular preferred embodiment, the present invention provided a composition comprising or corresponding to a Dimocapus extract for use in the treatment of influenza virus infections and/or at least one symptom thereof).

In another particular preferred embodiment, the present invention provides a composition comprising or corresponding to a Dimocapus extract for use in the prevention of influenza virus infection and/or at least one symptom thereof.

The symptoms of influenza virus infection referred to above comprise one or more of fever, chills, cough, congestion or runny nose, fatigue, general weakness, muscle or body aches, sore throat, diarrhea, nausea or vomiting, headache and sweating.

More severe symptoms and manifestations of influenza virus infection comprise primary influenza viral pneumonia, superimposed bacterial pneumonia (e.g. by Pneumococcus, Staphylococcus or Haernophilus influenzae) as well as exacerbations of chronic lung diseases (such as COPD). The involvement of further organs may lead myositis and rhabdomyolysis, encephalitis or myocarditis.

The subject or patient to be treated in accordance with the present invention may be an animal (e.g., a non-human animal). Preferably, the subject/patient is a mammal. More preferably, the subject/patient is a human (e.g., a male human or a female human) or a non-human mammal (such as, e.g., a guinea pig, a hamster, a rat, a mouse, a rabbit, a dog, a cat, a horse, a monkey, an ape, a marmoset, a baboon, a gorilla, a chimpanzee, an orangutan, a gibbon, a sheep, cattle, a pig, or a mink). Most preferably, the subject/patient to be treated in accordance with the invention is a human.

The term “treatment” of a disorder or disease as used herein (e.g., “treatment” of COVID-19) is well known in the art.

“Treatment” of a disorder or disease implies that a disorder or disease is suspected or has been diagnosed in a patient/subject. A patient/subject suspected of suffering from a disorder or disease typically shows specific clinical and/or pathological symptoms which a skilled person can easily attribute to a specific pathological condition (i.e. diagnose a disorder or disease).

The “treatment” of a disorder or disease may, for example, lead to a halt in the progression of the disorder or disease (e.g., no deterioration of symptoms) or a delay in the progression of the disorder or disease (in case the halt in progression is of a transient nature only). The “treatment” of a disorder or disease may also lead to a partial response (e.g., amelioration of symptoms) or complete response (e.g., disappearance of symptoms) of the subject/patient suffering from the disorder or disease. Accordingly, the “treatment” of a disorder or disease may also refer to an amelioration of the disorder or disease, which may, e.g., lead to a halt in the progression of the disorder or disease or a delay in the progression of the disorder or disease. Such a partial or complete response may be followed by a relapse. It is to be understood that a subject/patient may experience a broad range of responses to a treatment (such as the exemplary responses as described herein above). The treatment of a disorder or disease may, inter alia, comprise curative treatment (preferably leading to a complete response and eventually to healing of the disorder or disease) and palliative treatment (including symptomatic relief).

The term “prevention” of a disorder or disease as used herein (e.g., “prevention” of COVID-19) is also well known in the art. For example, a patient/subject suspected of being prone to suffer from a disorder or disease may particularly benefit from a prevention of the disorder or disease. The subject/patient may have a susceptibility or predisposition for a disorder or disease, including but not limited to hereditary predisposition. Such a predisposition can be determined by standard methods or assays, using, e.g., genetic markers or phenotypic indicators. It is to be understood that a disorder or disease to be prevented in accordance with the present invention has not been diagnosed or cannot be diagnosed in the patient/subject (for example, the patient/subject does not show any clinical or pathological symptoms). Thus, the term “prevention” comprises the use of a compound of the present invention before any clinical and/or pathological symptoms are diagnosed or determined or can be diagnosed or determined by the attending physician.

### Testing of the Composition Comprising a Dimocarpus Extract According to the Present Invention

The present invention uses standardized human 3D respiratory models as described in Zaderer et al Cells 2019; 8:1292 and Chandorkar et al. Sci Rep 2017; 7:11644 as a screening platform/in vitro model for assessing the effects of local application of the Dimocarpus extract/composition according to the present invention.

The model consists of in vitro reconstituted human primary normal bronchial or small airway epithelial (NHBE, SAE) cells from the upper and lower respiratory tract. The cells are seeded on transwell filters in collagen or cellulose-scaffold and cultured in Air-Liquid-Interphase (ALI).

Normal human bronchial epithelial (NHBE, Lonza, catalog no. CC-2540S) cells routinely cultured in air-liquid interphase (ALI) as described previously Zaderer et al Cells 2019; 8:1292 and Chandorkar et al. Sci Rep 2017; 7:11644). Briefly, cells were cultured in a T75 flask for 2 to 4 days until they reached 80% confluence. The cells were trypsinized and seeded onto GrowDexT (UPM)-coated 0.33-cm² porous (0.4-μm) polyester membrane inserts with a seeding density of 1×10⁵ cells per Transwell (Costar, Corning, NY, USA). The cells were grown to near-confluence in submerged culture for 2 to 3 days in specific epithelial cell growth medium according to the manufacturer's instructions. Cultures were maintained in a humidified atmosphere with 5% CO₂ at 37° C. and then transferred to ALI culture. The epithelium was expanded and differentiated using airway medium from Stemcell. The number of days in development was designated relative to initiation of ALI culture, corresponding to day 0. MucilAir nasal cells were obtained from Epithelix-Sarl (Suisse), Geneva, Switzerland, and cultured according to the manufacturer's protocol.

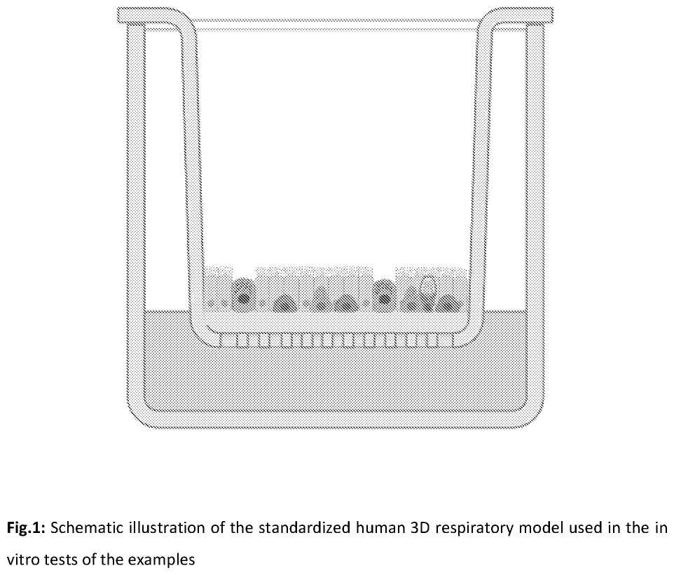

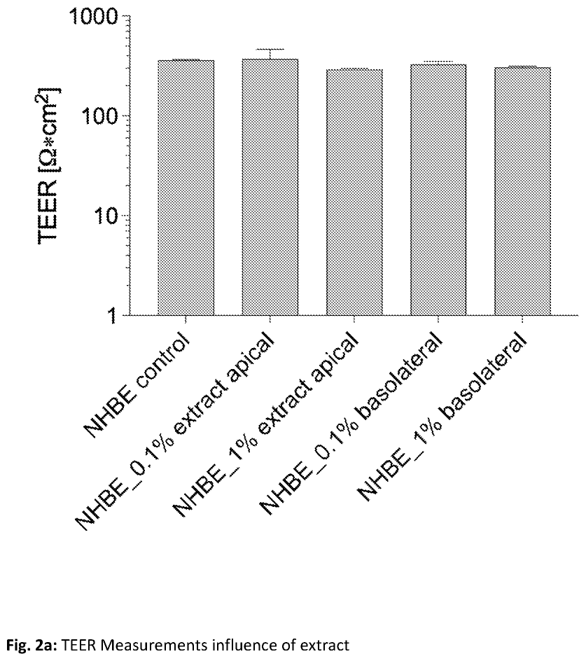

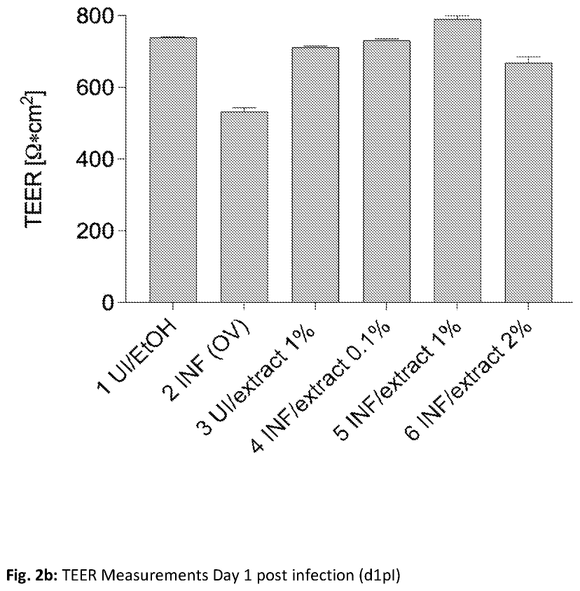

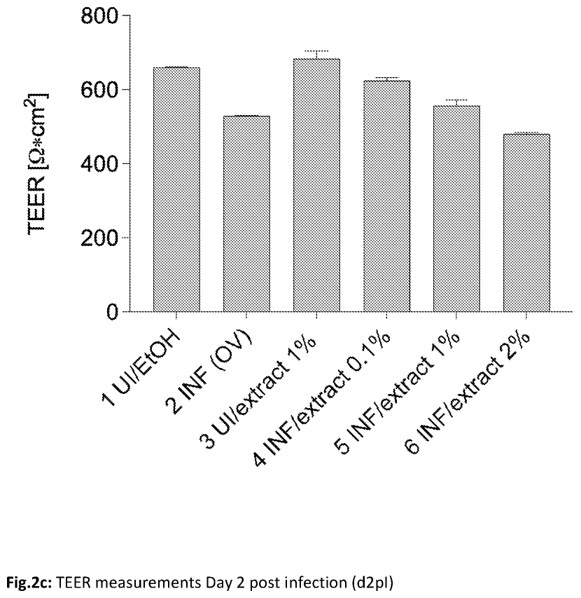

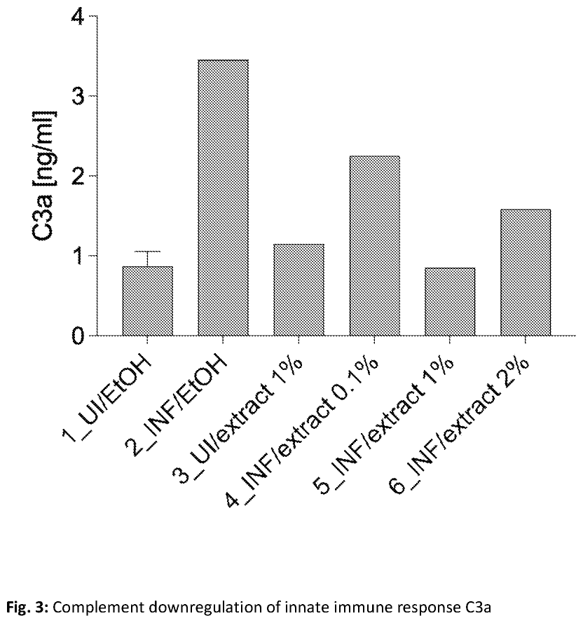

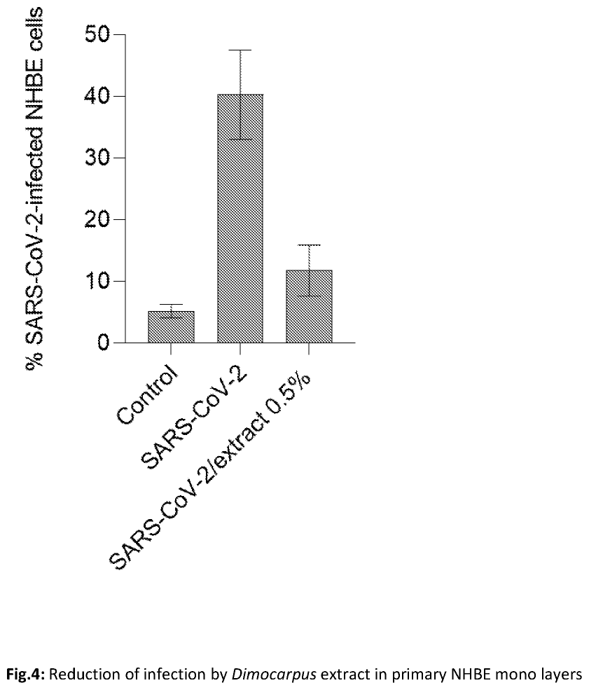

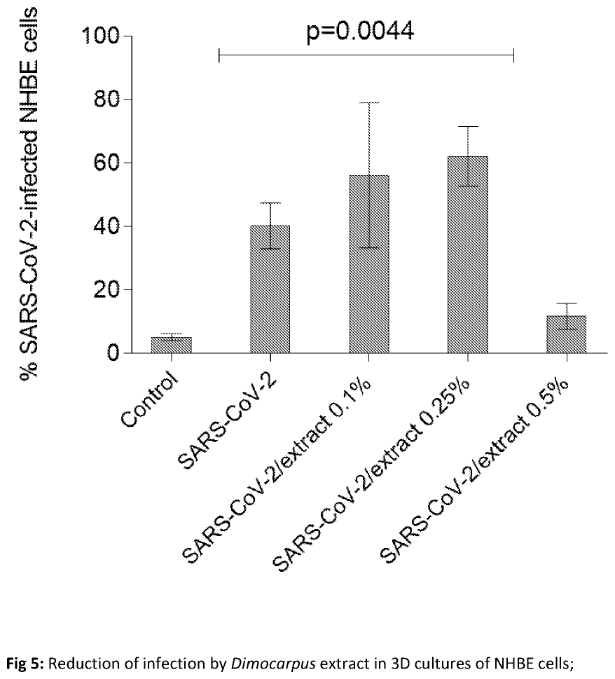

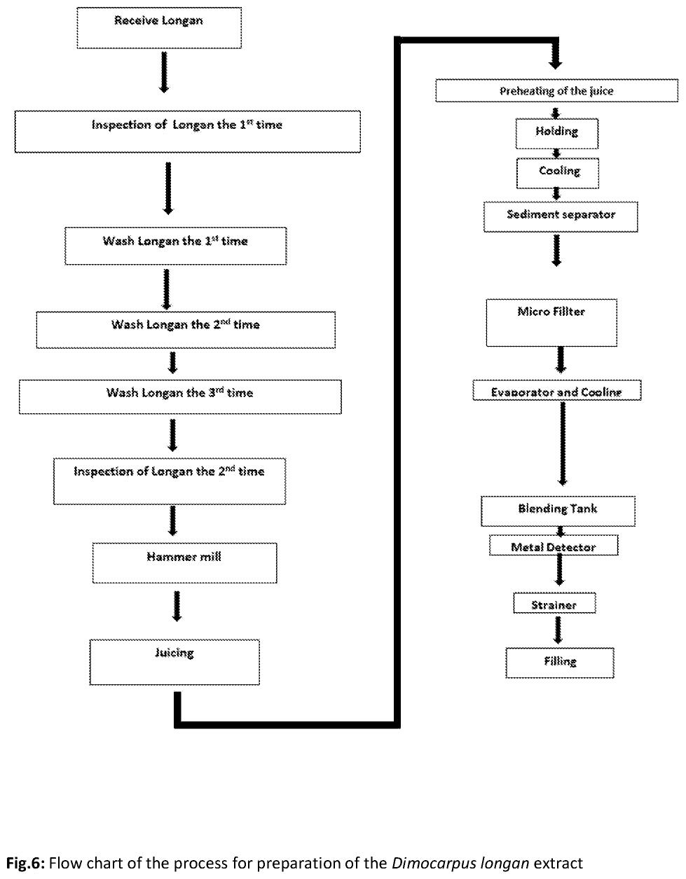

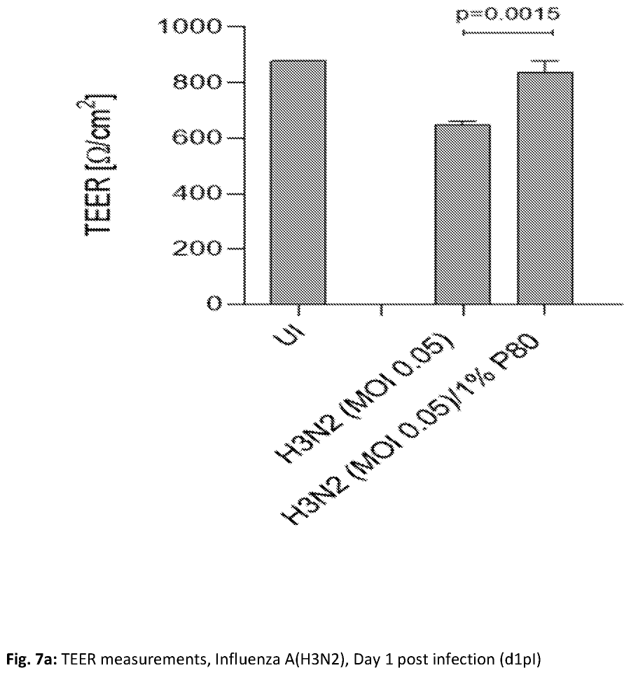

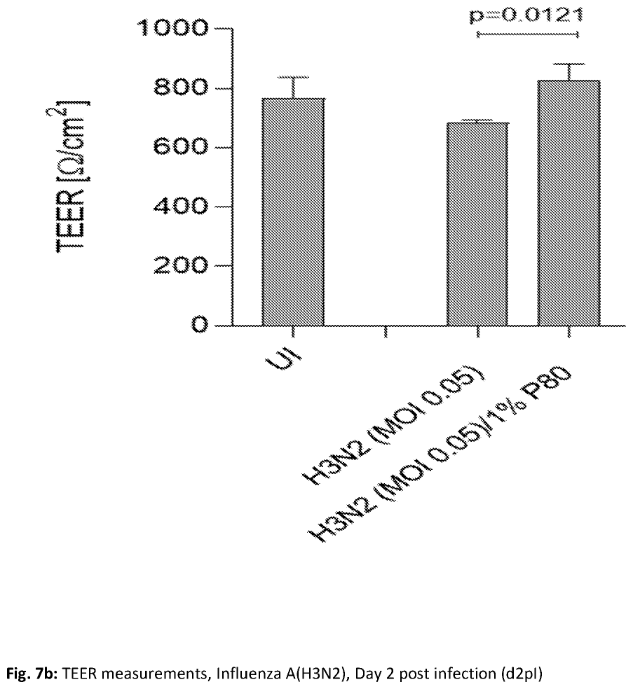

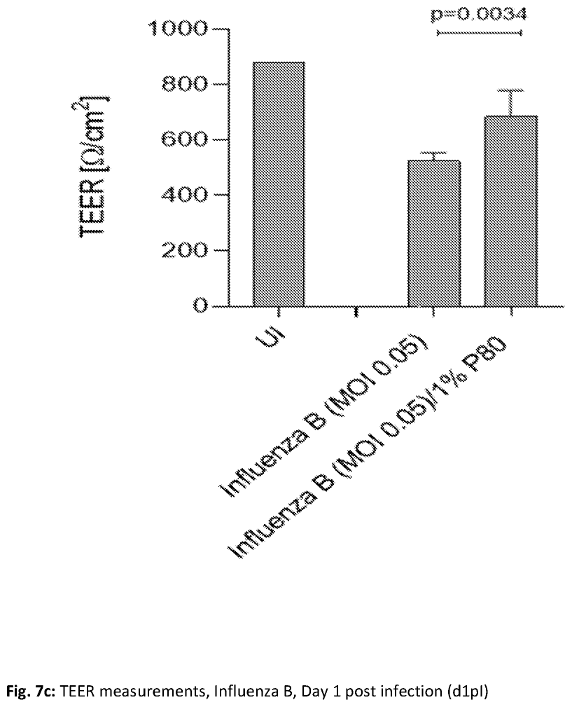

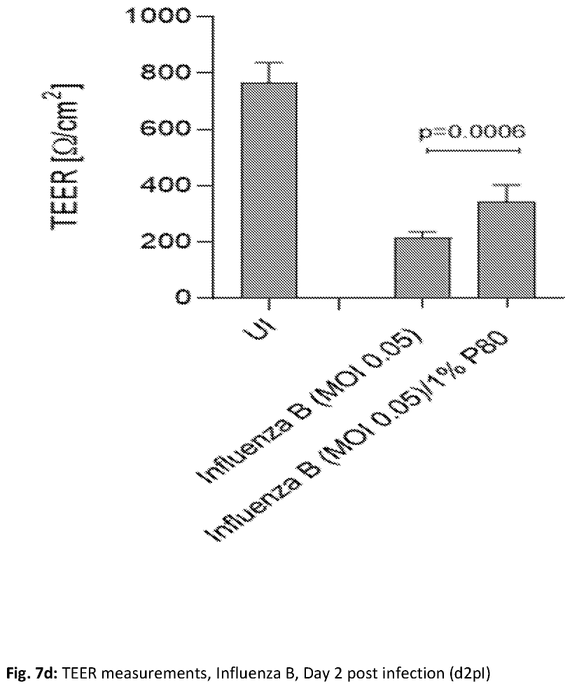

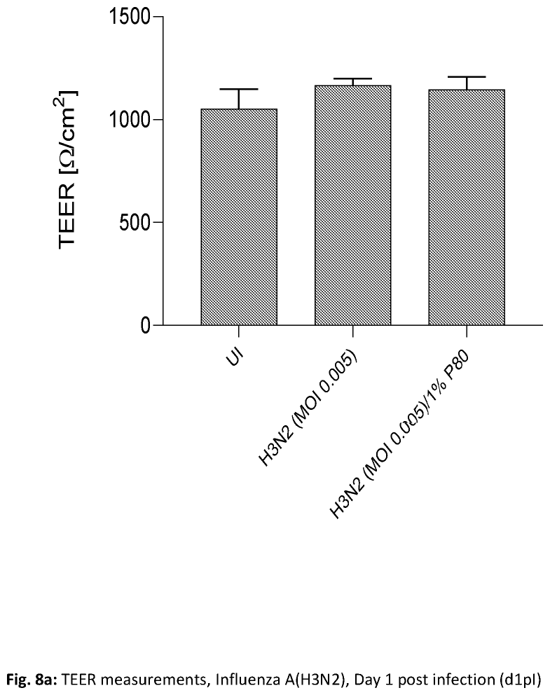

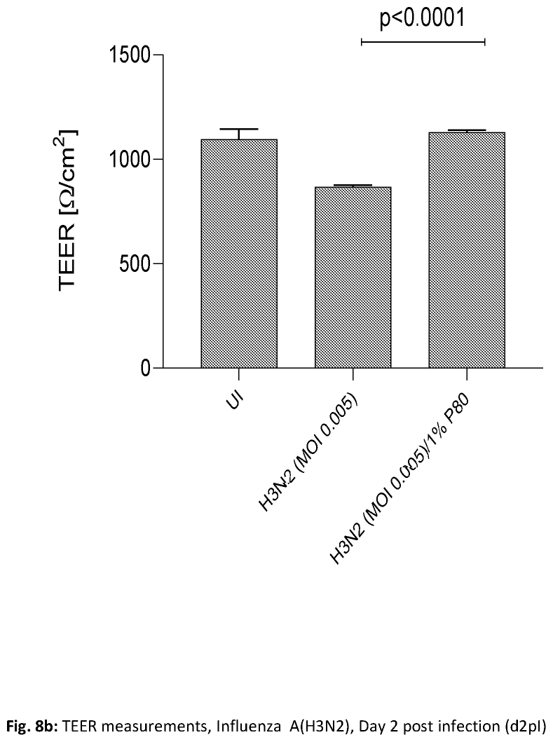

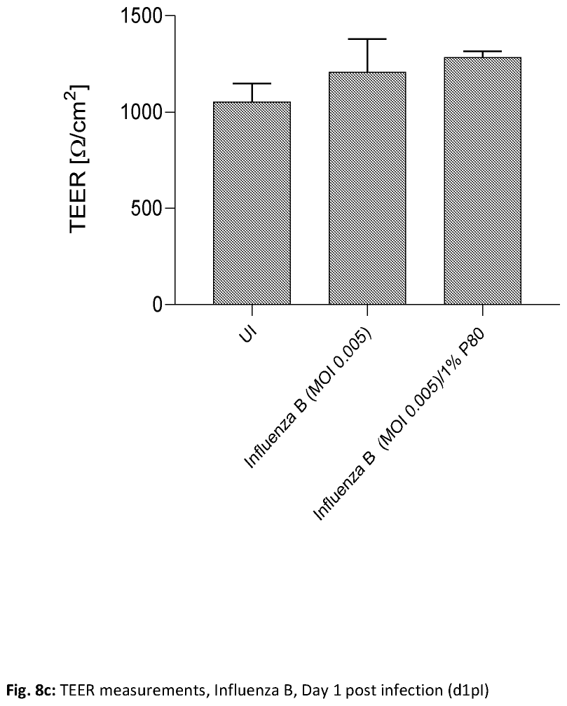

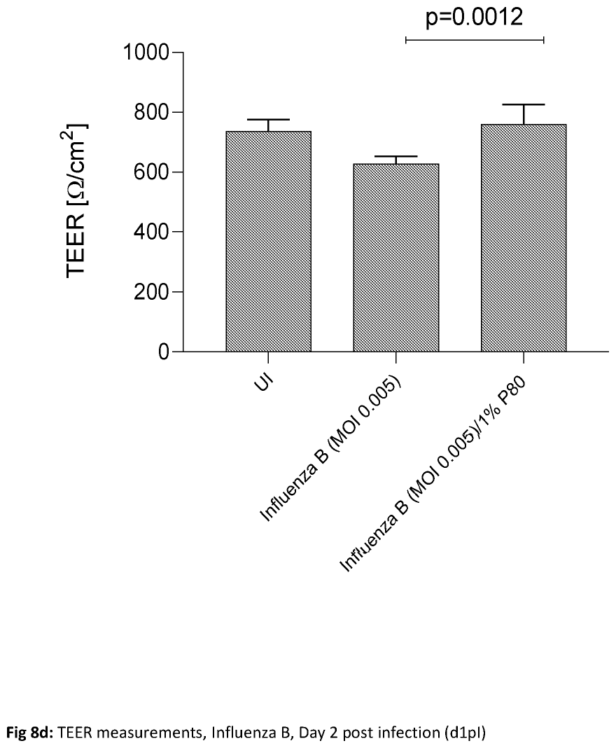

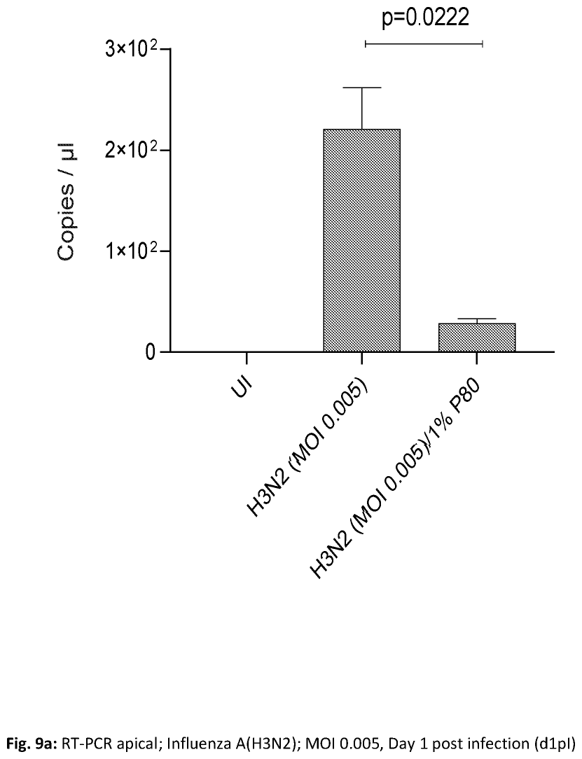

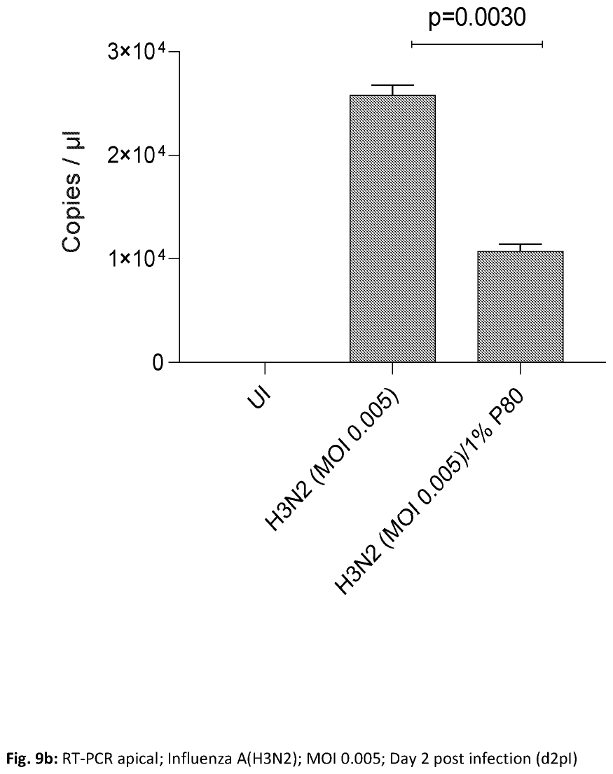

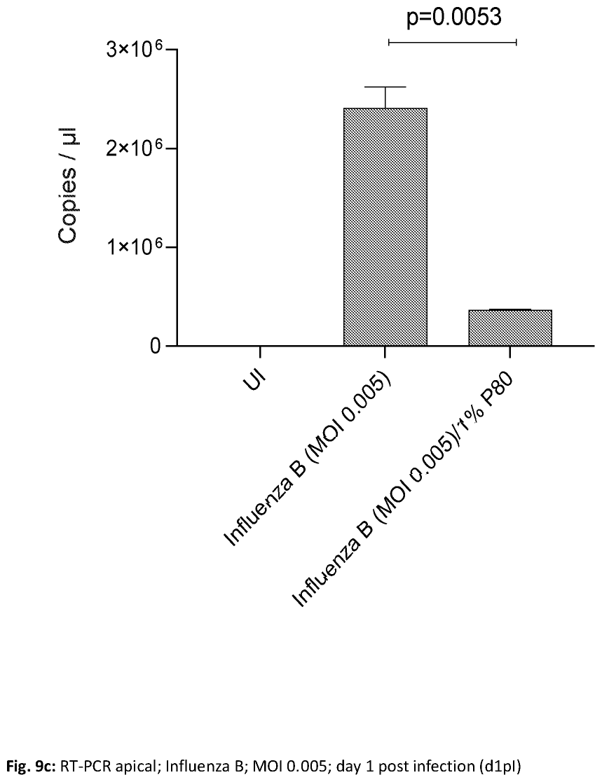

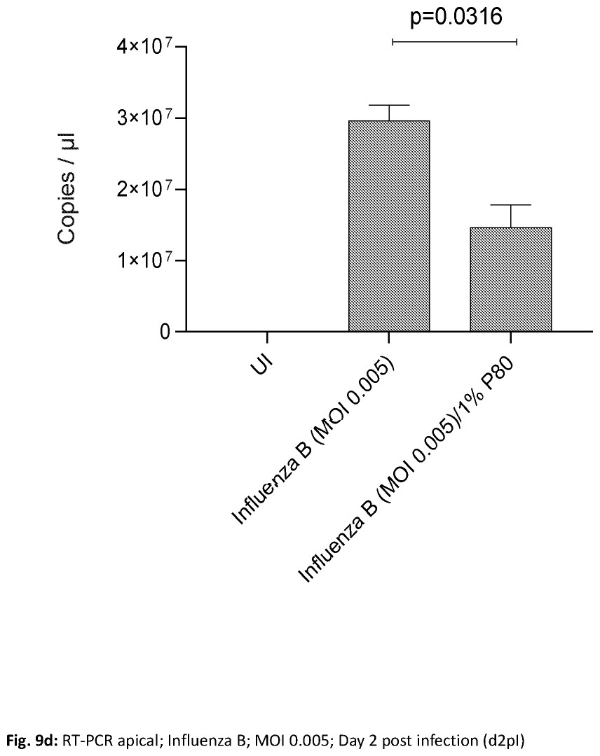

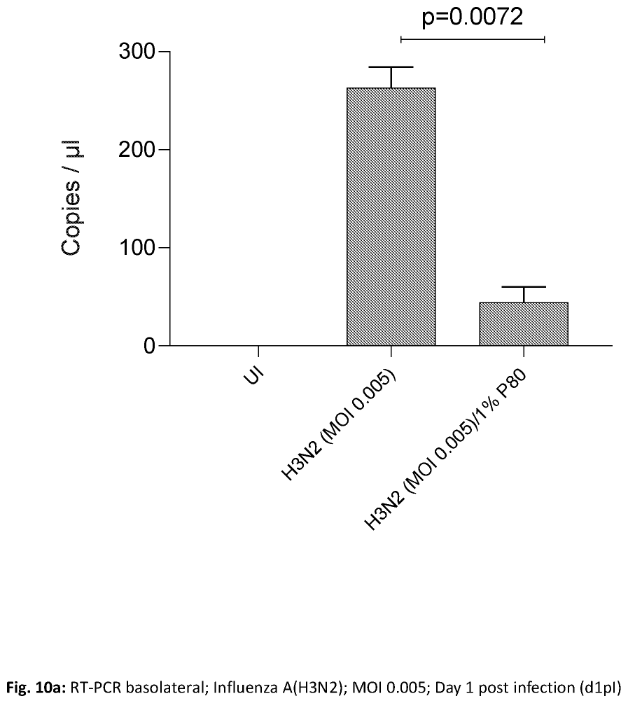

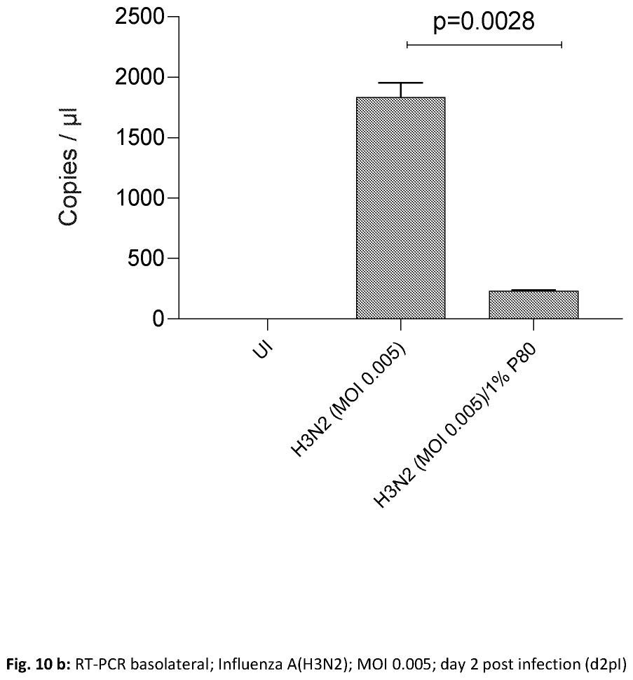

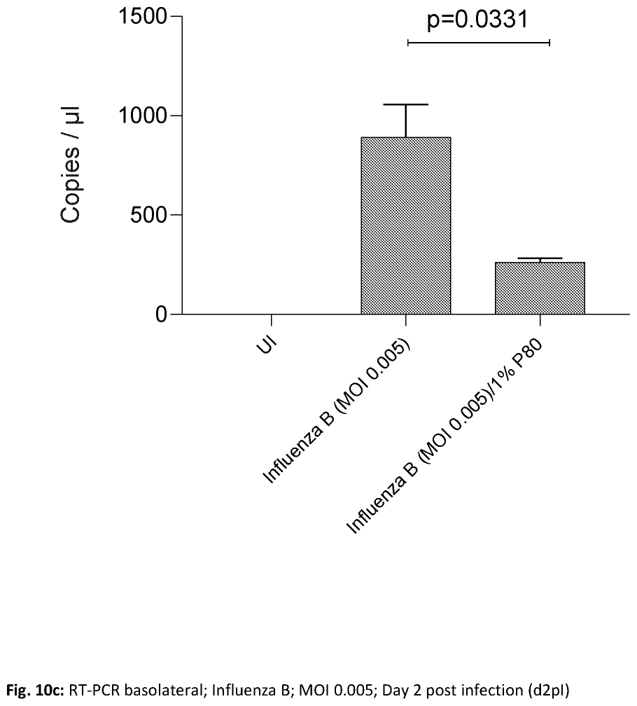

For illustrative purposes please be referred to FIG. 1.

In particular the following parameters have been analyzed using this in vitro model
mucuciliary clearance (MCC)
cilia beating frequency
apical cytokine release
transepithelial electrical resistance (TEER)
innate immune response (C3a)
detection of infection rate by imaging

### Experimental Examples

All experimental data were generated with a Dimocarpus extract in accordance with the present invention, in particular as disclosed in Table 1 above diluted with sterile double distilled water or physiological saline to the desired concentration.

### Example 1: Mucociliary Transport/Clearance (Prevention)

Study of the effects of Dimocarpus extract on cilia beating and mucociliary clearance in 3D NHBE cultures

Evaluation of cilia beating and mucociliary clearance aftertreatment with D-PBS spray control) or 1% Dimocarpus extract spray.

Mucus in the respiratory system is translocated within the mucosa by ciliary beating, which is an important non-specific defense mechanism called mucociliary clearance (MCC).

MCC is the main self-clearing system of the nasal cavity and paranasal sinuses and a very important means of non-specific defense against continuous organic and inorganic contamination conveyed by air. It works by trapping particles and microorganisms in the mucus and then by transporting the mucous film to the pharynx where it is eliminated with a cough or swallowed.

### Method

D-PBS (control) or 1% (w/w) Dimocarpus extract in D-PBS were sprayed onto fully differentiated 3D NHBE cultures (passage 2, day 92 in Air-Liquid-Interphase) (see FIG. 1). Cilia beating was assessed by brightfield analyses using the Operetta CLS (Perkin Elmer) HCS. Mucociliary clearance was monitored after adding fluorescently labeled beads (Invitrogen) to the fully differentiated 3D NHBE cultures (passage 2, day 92 in Air-Liquid-Interphase) (control or pre-treated with 1% Dimocarpus extract) and tracking the fluorescently labeled beads using the Harmony 4.8 software (Perkin Elmer) and Ready Made Solution (RMS) Bead Tracking (modified from RMS Cell Migration). Short videos have been recorded.

### Results

The results are summarized in Tables 2 and 3, below.

TABLE 2
Control- Dulbecco's Phosphate Buffered Saline (DPBS)
Population: Tracked beads Value
Number of objects 724

| Property | Mean | CV % | StdDev | Median | Max | Min | Sum |
|---|---|---|---|---|---|---|---|
| Number of | 13.0041 | 135.795 | 17.6589 | 5 | 60 | 1 | 9415 |
| Timepoints | | | | | | | |
| Duration [s] | 24.413 | 147.072 | 35.9047 | 8.11 | 120.01 | 0 | 17675 |
| Generation | 1.30249 | 42.4242 | 0.552569 | 1 | 4 | 1 | 943 |
| Accumulated d | 12.0511 | 140.263 | 16.9032 | 4.98738 | 103.174 | 0 | 8725 |
| Distance [μm] | | | | | | | |
| Displacement | 4.70871 | 200.944 | 9.46189 | 1.79784 | 79.1414 | 0 | 3409.11 |
| [μm] | | | | | | | |
| Speed [μm/s] | 0.786939 | 130.433 | 1.02643 | 0.421019 | 11.2083 | 0 | 546.136 |
| Straightness | 0543288 | 69.1173 | 0.375506 | 0.475041 | 1 | 0 | 349.334 |

TABLE 3
1% (w/w) Dimocarpus extract
Population: Tracked beads Value
Number of objects 1373

| Property | Mean | CV % | StdDev | Median | Max | Min | Sum |
|---|---|---|---|---|---|---|---|
| Number of | 9.88857 | 123.213 | 12.184 | 5 | 60 | 1 | 13577 |
| Timepoints | | | | | | | |
| Duration [s] | 18.0167 | 137.105 | 24.7017 | 8.097 | 119.59 | 0 | 24737 |
| Generation | 1.37291 | 60.0031 | 0.823786 | 1 | 9 | 1 | 1885 |
| Accumulated | 18.1506 | 122.163 | 22.1733 | 9.09017 | 169.732 | 0 | 24920.7 |
| Distance [μm] | | | | | | | |
| Displacement | 14.0176 | 137.187 | 19.2304 | 6.48222 | 168.232 | 0 | 19246.2 |
| [μm] | | | | | | | |
| Speed [μm/s] | 1.3152 | 68.5953 | 0.9017 | 1.09069 | 6.95387 | 0 | 1722.02 |
| Straightness | 0.808072 | 32.8567 | 0.265506 | 0.947254 | 1 | 0 | 1043.22 |

### CONCLUSION

The above data establish that apical application of Dimocarpus extract on epithelial cells enhances cilia speed and movement, thereby clearing the mucosa from viruses, reducing the virus load on the mucosa, indicating that the Dimocarpus extract may be particularly useful/important in the prevention of viral infection via the mucosa.

### Example 2: Transepithelial Electrical Resistance (TEER)

Study of the transepithelial electrical resistance (TEER) in 3D cultures of NHBE cells exposed to Dimocarpus extract

Transepithelial/transendothelial electrical resistance (TEER) is a widely accepted quantitative technique to measure the integrity of tight junction dynamics in cell culture models of epithelial and endothelial monolayers. TEER values are strong indicators of the integrity of the cellular barriers before they are evaluated for transport of drugs or chemicals. TEER measurements can be performed in real-time without cell damage and generally are based on measuring ohmic resistance or measuring impedance across a wide spectrum of frequencies. TEER measurements for various cell types have been reported with commercially available measurement systems—the present inventors used the EVOM2 Volt-Ohmmeter (World Precision Instruments, WPI). Determination of transepithelial electrical resistance is a simple and convenient technique that provides information about the uniformity of the Caco-2 cell layer on the filter support, and the integrity of the tight junctions formed between the polarized cells. Thus, TEER measurements may be used to study epithelial barrier function.

### Example 2(a)

Influence of different concentrations of the Dimocarpus extract on TEER of uninfected cells (control)

### Experimental Procedures

One puff of Dimocarpus longan spray (0.1% or 1% w/w in sterile double distilled water, corresponding to about 50 μl) was applied to the apical or the basolateral side of the fully differentiated epithelia (3D NHBE cultures), respectively. Transepithelial electrical resistance (TEER) values were measured in ALI culture using EVOM Volt-Ohmmeter with STX-2 chopstick electrodes (World Precision Instruments, Stevenage, UK).

For measurements, 0.1 ml and 0.7 ml of medium was added to the apical and basolateral chambers, respectively. Cells were allowed to equilibrate before TEER was measured. TEER values reported were corrected for the resistance and surface area of the Transwell filters. The results in Table 4 demonstrate that the extract has little influence on TEER of uninfected NHBE (see also FIG. 2(a)).

TABLE 4

| | 1 | 2 | 3 | 4 | 5 | 6 |
|---|---|---|---|---|---|---|
| NBHE control | 358 | 362 | 363 | 354 | 358 | 352 |
| NHBE 0.1% | 285 | 287 | 284 | 460 | 452 | 458 |
| apical | | | | | | |
| NHBE | 279 | 288 | 283 | 297 | 301 | 298 |
| 1% apical | | | | | | |
| NHBE 0.1% | 343 | 338 | 339 | 346 | 348 | 346 |
| basolateral | | | | | | |
| NHBE 1% | 305 | 311 | 308 | 315 | 309 | 311 |
| basolateral | | | | | | |

### Example 2(b) and (c)

Influence of different concentrations of the Dimocarpus extract on TEER of uninfected cells (day 1 post infection (b) and day 2 post infection (c))

### Experimental Procedures

One puff of Dimocarpus spray (0.1%, 1% or 2% w/w in sterile double distilled water, corresponding to about 50l) was applied to the apical side of the fully differentiated epithelia (3D NHBE cultures), prior to infection using SARS-CoV-2. The apical application was carefully performed to not mechanically disrupt the epithelial surface.

Transepithelial electrical resistance (TEER) values were measured using EVOM Volt-Ohmmeter with STX-2 chopstick electrodes (World Precision Instruments, Stevenage, UK). Measurements on cells in ALI culture infected or not with SARS-CoV-2 were taken immediately before the medium was exchanged. For measurements, 0.1 mL and 0.7 mL of medium was added to the apical and basolateral chambers, respectively. Cells were allowed to equilibrate before TEER was measured. TEER values reported were corrected for the resistance and surface area of the Transwell filters TEER was measured on day 1 post infection (d1pI) and day 2 post infection (d2pI).

Significantly lower TEER values were measured in SARS-CoV-2-infected epithelia on d5pl and d2pI compared to UI and Dimocarpus extract/UI.

The results in tables 5 and 6, below (see also FIGS. 2(b) and (c) demonstrate that Dimocarpus extract was able to rescue the TEER values in infected cultures at all concentrations tested on d1pI, (see FIG. 2b) and when applied as 0.1% spray also on d2pI (see FIG. 2c). Dimocarpus extract, however, significantly lowered TEER values of infected epithelia when applied as 2% solution indicating infection and destruction of epithelia.

TABLE 5
TEER (d1pl)

| | 1 | 2 | 3 |
|---|---|---|---|
| UI (unifected/EtOH) | 738 | 740 | 739 |
| INF (Infected with cell | 540 | 525 | 530 |
| culture isolate from | | | |
| CoVid-19 positive | | | |
| patient (dil | | | |
| 1/100/EtOH | | | |
| UI/Dimocarpus | 709 | 714 | 710 |
| extract 1% (control) | | | |
| INF/Dimocarpus | 728 | 734 | 728 |
| extract 0.1% | | | |
| INF/Dimocarpus | 797 | 783 | 787 |
| extract 1% | | | |
| INF/Dimocarpus | 650 | 680 | 676 |
| extract 2% | | | |

TABLE 6
TEER (d2pl)

| | 1 | 2 | 3 |
|---|---|---|---|
| UI (unifected/EtOH) | 660 | 661 | 658 |
| INF (Infected with cell | 530 | 528 | 527 |
| culture isolate from | | | |
| CoVid-19 positive | | | |
| patient (dil | | | |
| 1/100/EtOH | | | |
| UI/Dimocarpus | 669 | 698 | 678 |
| extract 1% (control) | | | |
| INF/Dimocarpus | 630 | 618 | 625 |
| extract 0.1% | | | |
| INF/Dimocarpus | 568 | 546 | 551 |
| extract 1% | | | |
| INF/Dimocarpus | 477 | 483 | 480 |
| extract 2% | | | |

### Example 3

Profiling of cytokines and anaphylotoxin also known as complement component C3a

This Assay allows for a laser-based identification of each biomarker and quantification of its amount in the sample. The levels of IL-la, IL-1ra, IL-6, IL-10, GM-CSF, P1-10, MCP-1, RANTES, TSLP, and TNF-α cytokines were measured with FLEXMAP-3D, a dual-laser, flow-based sorting and detection platform (Luminex, Austin, Tex). Supernatants of HAE cells treated with C5aR and/or SARS-CoV-2 were analyzed, using Magnetic Luminex Multiplex Assay (LXSAHM) from R&D Systems (Minneapolis, Minn), according to the manufacturer's instructions. Final data calculation and analysis was performed in Excel. C3a secretion of HAE tissue models was detected by the BD OptEIA Human C3a ELISA Kit (BD Biosciences) according to the manufacturer's instructions.

### Example 3a: Complement Down Regulation of Innate Immune Response C3a

Study of the effects of Dimocarpus extract on anaphylatoxin production (C3adesArg, C5adesArg) in SARS-CoV-2-infected 3D NHBE cultures.

Early events occurring directly after SARS-CoV-2 transmission to respiratory tissues can influence the outcome in the context of disease severity—in some patients, infection with COVID-19 results in excessive activation of the immune response at epithelial/immune barriers and the generation of a pro-inflammatory milieu. The development of a cytokine storm and acute lung injury, causing acute respiratory distress syndrome (ARDS), are potential undesirable consequences of the disease. ARDS accompanied by systemic coagulopathy are critical aspects of morbidity and mortality in COVID-19. These overshooting immune responses triggered by incoming viruses result in extensive tissue destruction during severe cases, resulting in tissue injury and multi-organ failure. Complement may be among the factors responsible for the immune overactivation, since complement deposition and high anaphylatoxin serum levels have been reported in patients with severe/critical disease.

Activation of the classical, alternate, or lectin complement pathways can result in the production of the C3a anaphylatoxin. C3a has been shown to be a multifunctional proinflammatory mediator. Thus, C3a has been shown to increase vascular permeability, to be spasmogenic and chemotactic, and to induce the release of pharmacologically active mediators from a number of cell types. C3a production in vivo may also initiate, contribute to, or exacerbate inflammatory reactions.

In blood plasma or serum, once formed, the nascent C3a anaphylatoxin is rapidly cleaved to the C3a-desArg form by the endogenous serum carboxypeptidase N enzyme. Thus, the quantitation of C3a-desArg in plasma or experimental samples should yield a reliable measurement of the level of complement activation that has occurred in the test samples under investigation.

### Experimental Procedures

Supernatants from non-infected and SARS-CoV-2 infected samples were collected after apical pretreatment with Dimocarpus extract 0.1%, 1% and 2% and also control cell supernatants (un-infected-NHBE UI, or infected with IBK isolate OV, NHBE-OV) were collected on day 2 post infection (d2pI), detergent treated for virus inactivation (2% Ipegal) and stored at −20° C.

Thus, the following samples were analyzed: 1_UI (uninfected)/EtOH, 2_INF (infected with cell-cultured isolate from a COVID19-positive patient (isolate OV) dil. 1/1000)/EtOH, 3_UI/1% (control), 4_INF/0.1%, 5_INF/1% and 6 INF/2% For Luminex analysis the collected supernatants were warmed to RT and 50 μl of each sample was processed following the manufacturer's protocol.

C3a in the samples was quantified using the BD OptEIA™ Human C3a ELISA Kit (Catalog No. 550499) for the in vitro quantitative determination of Human C3a-desArg in human EDTA plasma, serum and other biological samples in accordance with manufacture's protocol. As expected from TEER and imaging analysis, a pro-inflammatory response was induced in SARS-CoV-2 infected epithelia, which was completely blocked by pre-treating the epithelia with a composition comprising 1% Dimocarpus extract prior to infection. Values for C3a were also lower in epithelia pre-treated with a spray comprising 0.1% and 2% Dimocarpus extract (see FIG. 3)

### Example 3b: Down Regulation of Inflammatory Markers and Chemo Attractants for Immune Cells

Cytokine release (inflammatory response) of primary normal human bronchial epithelial (NHBE) cells

### Experimental Procedures

The expression of 10-pro-inflammatory cytokine/biomarkers (MCP-1, IP-10, IL-alpha, IL-6, TNF-alpha, RANTES, GM-CSF, IL-1ra, IL-10 and TSLP) was monitored using Human Magnetic Luminex Assay 10-plex human 2STD (R&D Systems). This assay allows for a laser-based identification of each biomarker and quantification of its amount in the sample. The level of all biomarkers in each sample was analyzed using a Luminex FLEXMAP 3D platform (SN-: FM3DD12269001), a dual laser, flow-based sorting and detection platform.

Supernatants from non-infected and SARS-CoV-2 infected samples were collected after apical or basolateral pretreatment with Dimocarpus Extract 0.1%, and also control cell supernatants (un-infected-NHBE UI, or infected with IBK isolate OV, NHBE-OV) were collected on day 2 post infection (d2pI), detergent treated for virus inactivation (2% Ipegal) and stored at −20° C. For Luminex analysis the collected supernatants were warmed to RT and 50 μl of each sample was processed following the manufacturer's protocol.

The results are summarized in Table 7, below and demonstrate that an anti-inflammatory activity could be observed. Release of MCP-1, RANTES and IL-6 was decreased in virus infected tissues treated with Dimocarpus extract compared to virus infected tissues without Dimocarpus extract-treatment.

TABLE 7
Quantification of cytokine levels from supernatants of3D NHBE cultures

| | TNF- | | | | | | | IL-1 | GM- | |
|---|---|---|---|---|---|---|---|---|---|---|
| Sample | alpha | IL-6 | IP-10 | IL-10 | MCP-1 | IL-1ra | RANTES | alpha | CFS | TSLP |
| Standard 6 | 235.5 | 145 | 71 | 224.25 | 90.75 | 101.5 | 405.25 | 36.5 | 108.5 | 111.25 |
| Standard 5 | 725.25 | 445.5 | 281.2 | 725 | 758.5 | 358.75 | 1712.25 | 144.5 | 341.75 | 339 |
| Standard 4 | 2209.75 | 1365.75 | 1215.25 | 2428.5 | 5663.25 | 1057.75 | 6811.75 | 538.75 | 1101 | 1044.5 |
| Standard 3 | 6722.75 | 4068.5 | 5282 | 7907 | 31770.75 | 3263.25 | 23079.25 | 2104.25 | 3391.5 | 3176.25 |
| Standard 2 | 18817.75 | 116345 | 20306 | 23683.25 | 86968.75 | 9087 | 60967.5 | 7119.25 | 10789.5 | 9937.25 |
| Standard 1 | 43923.25 | 28371 | 52604.5 | 59899.75 | 119640.8 | 19755.25 | 106128.8 | 17108.25 | 26850.25 | 25153.25 |
| NHBE UI | 15 | 282 | 42.5 | −6 | 743 | 362.5 | 13 | 8.5 | 102.5 | 827 |
| NHBE OV | 14 | 301 | 68.5 | −4 | 585.5 | 541.5 | 24.5 | 11.5 | 80 | 477.5 |
| NHBE 0.1% | −1 | 72 | 23 | −1 | 65 | 234.5 | 13 | −2.5 | 35 | 625 |
| DE apical | | | | | | | | | | |
| NHBE | 14 | 188.5 | 47 | −7 | 377 | 515.5 | 4 | 8.5 | 81 | 246.5 |
| 0.1% DE | | | | | | | | | | |
| apical/OV | | | | | | | | | | |
| NHBE 0.1% | 10 | 210 | 44.5 | −6.5 | 301 | 522.5 | 5.5 | 7.5 | 61.5 | 1050 |
| DE | | | | | | | | | | |
| basolateral | | | | | | | | | | |
| NHBE 0.1% | 20 | 268.5 | 57 | 15 | 362.5 | 401.5 | 14 | 0.5 | 107 | 560.5 |
| DE | | | | | | | | | | |
| basolateral/ | | | | | | | | | | |
| OV | | | | | | | | | | |

### Example 4

Visualization of SARS-CoV-2 infection and Reduction of infection by Dimocarpus extract

To visualize SARS-CoV-2 infection in monolayers and 3D tissue models, cells were infected with clinical specimens of SARS-CoV-2 and analyzed for characteristic markers in binding experiments after 2 h or for infection experiments on day 3 post-infection (d3pI). After SARS-CoV-2 exposure, 3D cell cultures were fixed with 4% paraformaldehyde. Intracellular staining was performed using 1× intracellular staining permeabilization wash buffer (10×; BioLegend, San Diego, CA, USA). Antibodies to stain the cell surface (wheat germ agglutinin [WGA-680]; ThermoFisher Scientific, Waltham, MA, USA), nuclei (Hoechst 33342; Cell Signaling Technologies, Danvers, MA, USA), actin (phalloidin-Alexa 647; Cell Signaling Technologies, Danvers, MA, USA), and complement C3 (C3-fluorescein isothiocyanate [FITC]; Agilent Technologies, Santa Clara, CA, USA) were used. Intracellular SARS-CoV-2 was detected using Alexa 594-labeled SARS-CoV-2 antibodies against S1 and N (both from Sino Biological, Beijing, China). The Alexa 594-labeling kit was purchased from Abcam, Cambridge, United Kingdom. After staining, 3D cultures were mounted in Mowiol. To study these complex models using primary cells cultured in 3D and to generate detailed phenotypic fingerprints for deeper biological insights in a high-throughput manner, the Operetta CLS system (PerkinElmer, Waltham, MA, USA) was applied. Spot analyses and absolute quantification for SARS-CoV-2-containing cells (Harmony software) were performed on more than 1,200 cells per condition.

On D2pI the cells were stained using nuclear counterstain Höchst Hoechst 33342 (Molecular Probes, H-3570, 1/1000), C3-FITC (Dako/Agilent, cat #F020102-2, 1/50), SARS-CoV-2-spike Antibody (Rabbit Mab, Sinobiological cat #40150-R007, 1/50) conjugated to Alexa488 or Alexa594 and Phalloidin-iFluor Alexa 647 (abcam, ab176759, 1/1000) for 3 hour after fixation (Cytofix, BD Biosciences, overnight) and permeabilization with Perm/Wash Buffer for intracellular staining (BD Biosciences, cat #554723, after staining the cells were washed with D-PBS, mounted on slides (Mowiol, 4-88, Carl Roth, #0718) and dried at RT overnight. Imaging was done using the Operetta CLS NHS (Perkin Elmer) and a 40× or 63× water objective. Imaging confirmed that as indicated in TEER measurement, SARS-CoV-2 infection destroyed respiratory epithelia already on d2pI compared to UI and Dimocarpus/UI.

The imaging demonstrated high infection and high innate immune activation (intracellular C3 induction) from respiratory epithelia infected with SARS-CoV-2.

Dimocarpus extract was able to rescue the epithelial integrity at 1% used and also blocked intracellular C3 generation (innate immune activation). The extract, however, worsened SARS-CoV-2 infection of respiratory epithelia when applied as 2% solution going along with epithelial destruction and C3 induction.

### Example 4 (a)

Study of the effect of different Dimocarpus extract concentrations on SARS-CoV-2 infection in primary NHBE monolayers

Primary normal human bronchial epithelial (NHBE) cells, (passage 3) p3 (25.000 cells/100p1) were seeded in an Operetta Cell Carrier Ultra 96-well plate.

After 3 days, ˜80% confluent NHBE cells were infected with various cell-cultured isolates from COVID19-positive patients for 5 days (5 dpl) or left uninfected (Control); isolate dilution: 1/1000; cells were in addition treated with Dimocarpus extract (0.5%-0.25%-0.1%) or vehicle.

Cells were stained using Höchst (nuclei, blue), C3 to detect intracellular complement formation (green), SARS-CoV-2-Spike 1 S1 and nucleocapsid (N) to detect productively infected cells (red), WGA (recognizes sugars on/in cells, orange) and brightfield (BF) images were also taken.

Imaging was done using the Operetta CLS HCS (Perkin Elmer) and the 63×Water Objective, images analyzed using the Harmony 4.8 software (Perkin Elmer).

A significant reduction of infection with SARS-CoV-2 was observed after treatment with 0.5% Dimocarpus extract (see FIG. 4).

### Example 4(b)

Imaging of Reduction of SARS-CoV-2 infection of NHBE cells in fully differentiated human 3D cultures by immunofluorescence
Infection of Dimocarpus extract treated NHBE cells

3D culture of NHBE cells grown and differentiated at air-liquid interphase (ALI) for at least 40 days.

The extract in was diluted in DPBS to obtain final concentrations 0.5%, 0.25% and 0.1%, respectively.

The cells were sprayed with the diluted extract as described above, as control served DPBS., The samples were incubated for 30 minutes at 37° C. and 5% Co₂.

To infect the cells 50 μl of viral dilutions were added on the apical side of each Transwell, for the untreated control the same amount of RPMI was added to the cells. The cells were then incubated at 37° C. and 5% CO₂ for a desired time period (overnight/1-3 days).

The infection rate was determined by confocal staining in accordance with the method described in Posch W, et al., J. Allergy Clin Immunol. 2021 June; 147(6):2083-2097 and Posch W, et al. mBio. 2021 Apr. 27; 12(2): e00904-21 using the Operetta CLS (Perkin Elmer) and the Harmony Software (also Perkin Elmer) for image analysis.

A significant reduction of infection with SARS-CoV-2 was observed after treatment with 0.5% Dimocarpus extract (see FIG. 5).

### Example 5

Measurement of tissue integrity with Trans Epithelial Electrical Resistance

As discussed above for example 2, Transepithelial/transendothelial electrical resistance (TEER) is a widely accepted quantitative technique to measure the integrity oftightjunction dynamics in cell culture models of endothelial and epithelial monolayers.

### Example 5 (a) and 5(b)

Effect of 1% Dimocarpus extract on TEER of cells infected with influenza virus A(H3N2), MOI 0.05, day 1 post infection (a) and day 2 post infection (b)

### Example 5(c) and (d)

Effect of 1% Dimocarpus extract on TEER of cells infected with influenza virus B, MOI 0.05, day 1 post infection (c) and day 2 post infection (d)

### Experimental Procedures

One puff of Dimocarpus spray (1% freshly diluted in double distilled water) corresponding to about 50 μl) was applied to the apical side of the fully differentiated epithelial cultures (3D NHBE cells, 80 days in ALI culture) prior to infection using influenza virus A(H3N2) and influenza virus B, respectively at a multiplicity of infection (MOI) of 0.05. The apical application was carefully performed to not mechanically disrupt the epithelial surface. The cells were incubated for one hour before infection with influenza viruses (influenza A and influenza B strains)

Transepithelial electrical resistance (TEER) values were measured using EVOM Volt-Ohmmeter with STX-2 chopstick electrodes (World Precision Instruments, Stevenage, UK). Measurements on cells in ALI culture infected or not with influenza virus (UI) were taken immediately before the medium was exchanged. For measurements, 0.1 mL and 0.7 mL of medium was added to the apical and basolateral chambers, respectively. Cells were allowed to equilibrate before TEER was measured. TEER values reported were corrected for the resistance and surface area of the Transwell filters TEER was measured on day 1 post infection (d1pI) and day 2 post infection (d2pI).

Significantly lower TEER values were measured in influenza A(H3N2) and influenza B-infected epithelia on d1pI and d2pI compared to Dimocarpus extract/UI.

The results depicted in FIG. 7a) to d) demonstrate that Dimocarpus extract was able to rescue the TEER values in infected cultures at the tested concentration of 1% on d1pI, (see FIGS. 7a and c) and on d2pl (see FIGS. 7b and d).

### Example 5 (e) and 5(f)

Effect of 1% Dimocarpus extract on TEER of cells infected with influenza virus A(H3N2), MOI 0.005, day 1 post infection (e) and day 2 post infection (f)

### Example 5(g) and (h)

Effect of 1% Dimocarpus extract on TEER of cells infected with influenza virus B, MOI 0.005, day 1 post infection (g) and day 2 post infection (h)

### Experimental Procedures

One puff of Dimocarpus spray (1% freshly diluted in double distilled water) corresponding to about 50 pl) was applied to the apical side of the fully differentiated epithelial cultures (3D NHBE cells, 80 days in ALI culture) prior to infection using influenza virus A(H3N2) and influenza virus B, respectively at a multiplicity of infection (MOI) of 0.005. The apical application was carefully performed to not mechanically disrupt the epithelial surface. The cells were incubated for one hour before infection with influenza viruses (influenza A and influenza B strains).

Transepithelial electrical resistance (TEER) values were measured using EVOM Volt-Ohmmeter with STX-2 chopstick electrodes (World Precision Instruments, Stevenage, UK). Measurements on cells in ALI culture infected or not with influenza virus (UI) were taken immediately before the medium was exchanged. For measurements, 0.1 mL and 0.7 mL of medium was added to the apical and basolateral chambers, respectively. Cells were allowed to equilibrate before TEER was measured. TEER values reported were corrected for the resistance and surface area of the Transwell filters TEER was measured on day 1 post infection (d1pI) and day 2 post infection (d2pl).

Significantly lower TEER values were measured in influenza A(H3N2) and influenza B-infected epithelia on d1pI and d2pl compared to Dimocarpus extract/UI.

The results depicted in FIG. 8a) to d) demonstrate that Dimocarpus extract was able to rescue the TEER values in infected cultures at the tested concentration of 1% on d1pI, (see FIGS. 8a and c) and on d2pl (see FIGS. 8b and d).

### Example 6

RT-PCR of apically and basolaterally released influenza virus particles to analyze the effect of Dimocarpus extract on the viral load of the epithelial cells
Determination of effect of 1% Dimocarpus extract on apically released influenza virus A (H3N2) particles by RT-PCR, day 1 post infection (a) and day 2 post infection (b)
Determination of effect of 1% Dimocarpus extract on apically released influenza virus B particles by RT-PCR, day 1 post infection (c) and day 2 post infection (d)
Determination of effect of 1% Dimocarpus extract on basolaterally released influenza virus A (H3N2) particles by RT-PCR, day 1 post infection (e) and day 2 post infection (f)
Determination of effect of 1% Dimocarpus extract on basolaterally released influenza virus B particles by RT-PCR, day 1 post infection (g) and day 2 post infection (h)

### Experimental Procedures

One puff of Dimocarpus spray (1% freshly diluted in double distilled water) corresponding to about 50 pl) was applied to the apical side of the fully differentiated epithelial cultures (3D NHBE, 80 days in ALI culture) prior to infection using influenza virus A(H3N2) and influenza virus B, respectively at a multiplicity of infection (MOI) of 0.005. The apical application was carefully performed to not mechanically disrupt the epithelial surface. The cells were incubated for one hour before infection with influenza viruses (influenza A and influenza B strains).

Release of virus particles (at the apical side and the basolateral side, respectively) was determined on day 1 post infection (d1pI) and day 2 post infection (d2pl) by RT-PCR.

### RNA Isolation:

For RNA Isolation from the viral particles the FavorPrep Viral RNA/Viral Nucleic Acid Mini Kit (#FAVNK001-2), (Favorgen Biotech) was used. According to manufacturer's instructions (User Manual) 140 μl of sample were mixed with 560 μl of VNE lysis buffer and further the protocol was performed as described by the company.

To generate samples for influenza infection assays (detection and quantification of viral RNA by R-PCR), 140 μl samples were harvested from the basolateral medium chamber ofTranswells in ALI state.

To generate RT-PCR samples from the apical side, the medium from TEER measurements was harvested (see examples 2, 7 and 8.)

### RNA Detection and Quantification by RT-PCR

The PCR was carried out using the LUNA Universal Probe One-Step RT-qPCR Kit E3006G (New England BioLabs Inc.) according to manufacturer's instructions using the following primers and probes (Metabion, Planegg, Germany):

H1N1/H3N2 metabion
MP-39-37-F(N2)-F
CCM AGG TCG AAA CGT AYG TTC TCT CTA TC
MP 183 153 R(N2)-R
TGA CAG RAT YGG TCT TGT CTT TAG CCA YTC CA
Probe-P
6-Fam-ATYTCG GCT TTG AGG GGG CCT BHQ (Probe)
Type B Victoria metabion
TypeB-Vic-F F
CCT GTT ACA TCT GGG TGC TTT CCT ATA ATG
TypeB-Vic-R R
GTT GAT ARC CTG ATA TGT TCG TAT CCT CKG
TypeB Vic-P P
6-FAM TTA GAC AGC TGC CTA ACC BHQ 1(Probe)

PCR STD copy number 10⁸-10⁴

| Name | Function | DNA-Sequence | SEQ ID NO: |
|---|---|---|---|
| MP-39-37-F(N2)-F | H1N1/H3N2 | CCM AGG TCG AAA | SEQ ID NO: 1 |
| | Forward primer | CGT AYG TTC TCT | |
| | | CTA TC | |
| MP 183 153 R(N2)-R | H1N1/H3N2 | TGA CAG RAT YGG | SEQ ID NO: 2 |
| | Reverse primer | TCT TGT CTT TAG | |
| | | CCA YTC CA | |
| Probe-P | FAM/BHQ-marked | ATY TCG GCT TTG | SEQ ID NO: 3 |
| | probe | AGG GGG CCT | |
| TypeB-Vic-F F | Type B (Victoria) | CCT GTT ACA TCT | SEQ ID NO: 4 |
| | Forward primer | GGG TGC TTT CCT | |
| | | ATA ATG | |
| TypeB-Vic-R R | Type B (Victoria) | GTT GAT ARC CTG | SEQ ID NO: 5 |
| | Reverse primer | ATA TGT TCG TAT | |
| | | CCT CKG | |
| TypeB Vic-P P | FAM/BHQ-marked | TTA GAC AGC TGC | SEQ ID NO: 6 |
| | probe | CTA ACC | |

The PCR results were analyzed using Bio-Rad CFX Manager or Bio-Rad Maestro Software.

Significantly lower copy numbers were measured apically in influenza A(H3N2) and influenza B-infected epithelia on d1pI and d2pl treated with Dimocarpus extract compared to untreated infected cultures.

The results depicted in FIG. 9a) to d) demonstrate that Dimocarpus extract was able to lower the apical viral load/the number of apically excreted virus particles from infected cultures at the tested concentration 1% on d1pI, (see FIGS. 9a and 9c) and on d2pl (see FIGS. 9b and 9d).

Significantly lower copy numbers were also measured basolaterally in influenza A(H3N2) and influenza B-infected epithelia on dipl and d2pl treated with Dimocarpus compared to untreated infected cultures.

The results depicted in FIG. 10a) to 10c) demonstrate that Dimocarpus extract was able to lower the basolateral viral load/the number of basolaterally excreted virus particles from infected cultures at the tested concentration 1% on d1pI, (see FIG. 10a) and on d2pl (see FIGS. 10b and 10c). The copy numbers for basolaterally excreted influenza virus B particles on d1pP were below the detection limit of 50 copies/pl).

From the above follows, that topical application of Dimocarpus extract of the present protected both the integrity of the tissue from influenza virus A and B infection and prevented the intracellular formation of new viral particles and their excretion.

Thus, the present inventors were able to demonstrate that topical application of a composition according to the present invention comprising Dimocarpus extract
exhibits an antiviral activity and decreases infection
interferes with binding of enveloped viruses to the surface of the mucosal epithelium and thereby prevents entry of enveloped viruses, such as SARS-CoV-2 into the lining of the epithelial cells of the (upper) respiratory tract.
down regulates pro-inflammatory cytokines and has a modulatory effect on the innate immune system, no destruction of lung tissue bycytokine storm, C3a, preventing tissue damage after infection
has a positive effect on the transport of (virus) particles by the cilia activity and the rhythmical beating, facilitates MCC by stimulating cilia movement
has a moistening effect While the present invention is explained herein with reference to particular embodiments, modifications and improvements obvious to those skilled in the art are included in the scope of the present invention.

The contents of all documents (patent documents and other references) cited in the present application are incorporated herein in their entirety by reference.

1. A method comprising administering a composition comprising Dimocarpus extract to a subject that has a respiratory infection with an enveloped virus, enveloped single stranded virus, enveloped positive single strand RNA virus (+ssRNA) virus, an influenza or a coronavirus.

2-3. (canceled)

4. The method according to claim 1, wherein the respiratory infection is an upper and/or lower respiratory tract infection.

5. The method according to claim 1, wherein the Dimocarpus extract is an extract of Dimocarpus longan Lour.

6. The method according to claim 1, wherein the virus is selected from an influenza virus, a respiratory syncytial virus (RSV), a human parainfluenza virus (HPIV), a human metapneumovirus (HPMV), a rhinovirus or a coronavirus.

7. The method according to claim 1, wherein the virus is selected from SARS-CoV, MERS-CoV and SARS-CoV-2.

8. The method according to claim 1, wherein the infection is a SARS-CoV-2 infection (COVID-19).

9. The method according to claim 1, wherein the virus is selected from a subtype of Influenza Virus type A and Influenza Virus type B.

10. The method according to claim 1, wherein the infection is an Influenza type A(H3N2) or Influenza Type B infection.

11. The method according to claim 1, wherein the Dimocarpus extract comprises vitamin C, one or more polyphenols selected from corilagin, gallic acid, ellagic acid, ellagic acid conjugates, (−)-epicatechin, quercetin, kaempferol, tannic acid (tannin) and chlorogenic acid; protocatechuic acid, brevifolin, γ-aminobutyric acid (GABA), carbohydrates and water.

12. The method according to claim 1 wherein the Dimocarpus extract comprises vitamin C, corilagin, gallic acid, ellagic acid, ellagic acid conjugates tannic acid, GABA, saccharose, glucose, fructose, polysaccharides and water.

13. The method according to claim 1, wherein the Dimocarpus extract comprises

| | | |
|---|---|---|
| Vitamin C | 100-1000 | mg/kg |
| Tannic acid | 200-700 | mg/kg |
| Gallic acid | 200-600 | mg/kg |
| Ellagic acid (incl. conj.) | 600-1200 | mg/kg |
| Corilagin | 750-2000 | mg/kg |
| GABA | 1200-2000 | mg/kg |
| Total phenolic content | 2950-7600 | mg/kg |
| Total carbohydrate | 700-800 | g/kg |
| Sucrose | 30-50% | w/w |
| Glucose | 5-25% | w/w |
| Fructose | 10-20% | w/w |
| Polysaccharides | 50-85 | g/kg |
| Water | 15-25% | w/w |

14. The method according to claim 1, wherein the Dimocarpus extract comprises

| | | |
|---|---|---|
| Vitamin C | 105-760 | mg/kg |
| Tannic acid | 420-510 | mg/kg |
| Gallic acid | 340-428 | mg/kg |
| Ellagic acid (incl. conj.) | 1010-1230 | mg/kg |
| Corilagin | 1188-1880 | mg/kg |
| GABA | 1133-1896 | mg/kg |
| Total phenolic content | 3500-7600 | mg/kg |
| Total carbohydrate | 700-800 | g/kg |
| Sucrose | 30-50% | w/w |
| Glucose | 7-23% | w/w |
| Fructose | 10-20% | w/w |
| Polysaccharides | 50-80 | g/kg |
| Water | 18-24% | w/w |

15. The method according to claim 1, wherein the Dimocarpus extract comprises Vitamin C 200-760 mg/kg

| | | |
|---|---|---|
| Vitamin C | 200-760 | mg/kg |
| Tannic acid | 420-510 | mg/kg |
| Gallic acid | 380-430 | mg/kg |
| Ellagic acid (incl. conj.) | 1010-1100 | mg/kg |
| Corilagin | 1200-1500 | mg/kg |
| GABA | 1150-1700 | mg/kg |
| Total phenolic content | 4000-7600 | mg/kg |
| Total carbohydrate | 700-800 | g/kg |
| Sucrose | 300-400 | g/kg |
| Glucose | 100-200 | g/kg |
| Fructose | 100-150 | g/kg |
| Polysaccharides | 50-70 | g/kg |
| Water | 19-23% | w/w |

16. The method according to claim 1, wherein the Dimocarpus extract comprises

| Vitamin C | 720 | mg/kg |
|---|---|---|
| Tannic acid | 430 | mg/kg |
| Gallic acid | 409 | mg/kg |
| Ellagic acid (incl. conj.) | 1050 | mg/kg |
| Corilagin | 1250 | mg/kg |
| GABA | 1638 | mg/kg |
| Total phenolic content | 7565 mg/kg (0.76 % w/w) | |
| Total carbohydrate | 742 | g/kg |
| Sucrose | 424 | g/kg |
| Glucose | 117 | g/kg |
| Fructose | 133 | g/kg |
| Polysaccharides | 66 | g/kg |
| Water | 21% | w/w |

17. The method according to claim 1, wherein the extract has as sugar content of 74 to 840 Brix, preferably 76 to 82° Brix and most preferably of 78 to 800 Brix.

18. The method according to claim 1, wherein the extract is produced from whole fresh fruits.

19. The method according to claim 1, wherein the Dimocarpus extract is obtainable by a method comprising the following steps:
(a) extraction of the Dimocarpus juice from whole fresh fruit, followed by
(b) separation of the solids from the obtained raw liquid;
(c) concentration of the liquid obtained in step (b) to obtain a sugar concentration of about 74 to 84, preferably 76 to 82 and most preferably to 78 to 80° Brix and
(d) aseptical packing.

20. The method according to claim 1, wherein the Dimocarpus extract is obtainable by a method comprising the following step(s):
(1) milling whole fresh Dimocarpus fruit;
(2) extracting raw fruit juice from the milled fruits obtained in step (1);
(3) Conditioning of the raw juice obtained in (2) by rapidly heating to about 95 −98° C., maintaining at about 95-98° C.; followed by rapid cooling to about 5-15° C.;
(4) Separation of the supernatant from the product of step (3) by centrifugation and microfiltration of the supernatant;
(5) concentration of the supernatant obtained in step (4), preferably by evaporation at reduced pressure to obtain a sugar concentration of about 74-84° Brix;
(6) microfiltration of the concentrate obtained in step (5); and
(7) optionally aseptical packing.

21. The method according to claim 1, wherein the composition is to be applied topically via the ocular, nasal or pharyngeal route.

22. The method according to claim 1, in form of eye drops, a mouthwash, gargle, nasal drops, nasal spray/aerosol, pharyngeal drops, or a pharyngeal spray/aerosol.

23. The method according to claim 1, in form of nasal drops, nasal spray/aerosol, pharyngeal drops or pharyngeal spray/aerosol.

24. A composition comprising Dimocarpus extract, wherein the Dimocarpus extract comprises vitamin C, one or more polyphenols selected from corilagin, gallic acid, ellagic acid, ellagic acid conjugates, (−)-epicatechin, quercetin, kaempferol, tannic acid (tannin) and chlorogenic acid; protocatechuic acid, brevifolin, γ-aminobutyric acid (GABA), carbohydrates and water.


SEQUENCE LISTING (6 sequences)

SEQ ID NO: 1 - DNA, 29 bp, synthetic construct
  source 1..29: mol_type = other DNA
  misc_feature 1..29: note = forward primer H1N1 / H3N2 metabion MP-39-37-F (N2)
ccmaggtcgaaacgtaygttctctctatc

SEQ ID NO: 2 - DNA, 32 bp, synthetic construct
  source 1..32: mol_type = other DNA
  misc_feature 1..32: note = reverse primer H1N1 / H3N2 metabion MP 183 153 R (N2)
tgacagratyggtcttgtctttagccaytcca

SEQ ID NO: 3 - DNA, 21 bp, synthetic construct
  source 1..21: mol_type = other DNA
  misc_feature 1..21: note = probe H1N1 / H3N2 metabion containing 5' 6-FAM and 3' BHQ
atytcggctttgagggggcct

SEQ ID NO: 4 - DNA, 30 bp, synthetic construct
  source 1..30: mol_type = other DNA
  misc_feature 1..30: note = forward primer Type B Victoria metabion
cctgttacatctgggtgctttcctataatg

SEQ ID NO: 5 - DNA, 30 bp, synthetic construct
  source 1..30: mol_type = other DNA
  misc_feature 1..30: note = reverse primer Type B Victoria metabion
gttgatarcctgatatgttcgtatcctckg

SEQ ID NO: 6 - DNA, 18 bp, synthetic construct
  source 1..18: mol_type = other DNA
  misc_feature 1..18: note = probe Type B Victoria metabion containing 5' 6-FAM and 3' BHQ
ttagacagctgcctaacc